(19) United States
(12) Patent Application Publication
(10) Pub. No.: US 2024/0368556 A1
(43) Pub. Date: Nov. 7, 2024

(54) LIVER ORGANOID MODEL FOR HYPERBILIRUBINEMIA AND METHODS OF MAKING AND USING SAME

(71) Applicant: CHILDREN’S HOSPITAL MEDICAL CENTER, Cincinnati, OH (US)

(72) Inventors: Takanori Takebe, Cincinnati, OH (US); Hasan Al Reza, Cincinnati, OH (US)

(21) Appl. No.: 18/568,764

(22) PCT Filed: Jun. 10, 2022
(86) PCT No.: PCT/US2022/033066

§ 371 Date: Dec. 8, 2023

Related U.S. Application Data
(60) Provisional application No. 63/209,908, filed on Jun. 11, 2021.

(51) Int. Cl. C12N 5/071

(52) U.S. Cl. CPC C12N 5/0671; C12N 2501/39; C12N 2510/00

(57) ABSTRACT

Disclosed herein are improved methods of maturing pluripotent stem cell-derived liver organoids with the use of hemoglobin metabolites such as bilirubin. Furthermore, by modulating the amounts of bilirubin used, liver organoids can be used as a model for hyperbilirubinemia. The scalability and tractability of these liver organoids made them excellent targets for drug screening against diseases such as hyperbilirubinemia. Also shown herein is the use of exogenous L-gulonolactone oxidase for improving viability of bilirubin-treated liver organoids.

### CROSS-REFERENCE TO RELATED APPLICATIONS

This application claims priority to and benefit of PCT Application No. PCT/US2022/033066, filed Jun. 10, 2022, and U.S. Provisional Patent Application No. 63/209,908, filed Jun. 11, 2021, each of which is herein incorporated by reference in its entirety.

### STATEMENT REGARDING FEDERALLY SPONSORED R&D

This invention was made with government support under DK128799-01 awarded by the National Institutes of Health. The government has certain rights to the invention.

### FIELD OF THE INVENTION

Aspects of the present disclosure relate generally to liver organoids and improved methods of maturing liver organoids to resemble a non-fetal state. Additional aspects relate to the generation of hyperbilirubinemia models.

### BACKGROUND

Hyperbilirubinemia, and associated jaundice, is the condition of elevated levels of bilirubin, which is a natural product of heme catabolismilirubin is filtered from the blood by the liver and is converted to water soluble intermediates, which are then released to the intestinal tract in bile, metabolized by microbiota, and excreted as waste. In neonates, bilirubin levels, which were originally cleared by the mother through the placenta, might not be adequately cleared by the immature liver. Excessive levels of bilirubin may potentially cause severe neurological damage (kernicterus). In adults, hyperbilirubinemia may also result from diseases affecting the liver, such as hepatitis and cirrhosis. Neonatal hyperbilirubinemia is treated by phototherapy, or with blood transfusion in extreme cases, whereas treatments in adults are directed to the underlying cause. There is a lasting need for additional treatments for hyperbilirubinemia and models for studying this condition.

### SUMMARY

Disclosed herein are methods of maturing a fetal-like liver organoid. In some embodiments, the methods comprise contacting a fetal-like liver organoid with a low/first concentration of bilirubin, thereby maturing the fetal-like liver organoid to a mature liver organoid. In some embodiments, the low/first concentration of bilirubin is a human fetal physiological concentration of bilirubin.

Also disclosed herein are methods of producing a hyperbilirubinemia liver organoid. In some embodiments, the methods comprise contacting a liver organoid with a high/second concentration of bilirubin, thereby forming the hyperbilirubinemia liver organoid.

Also disclosed herein are methods of treating hyperbilirubinemia in a subject in need thereof. In some embodiments, the methods comprise administering a glucocorticoid antagonist to the subject in need thereof.

Also disclosed herein are the mature liver organoid compositions disclosed herein, such as those made by the methods disclosed herein.

Also disclosed herein are the hyperbilirubinemia liver organoid compositions disclosed herein, such as those made by the methods disclosed herein.

Also disclosed herein are liver organoids comprising a functional GULO protein and/or a gene or mRNA, or both, that encodes for the functional GULO protein, whereby the liver organoids are able to synthesize ascorbate.

Also disclosed herein are liver cells comprising a functional GULO protein and/or a gene or mRNA, or both, that encodes for the functional GULO protein, whereby the liver cells are able to synthesize ascorbate.

Also disclosed herein are methods of increasing bilirubin conjugation and metabolism in a liver cell. In some embodiments, the methods comprise expressing a functional GULO protein and/or a gene or mRNA, or both, that encodes for the functional GULO protein, whereby the liver cell is able to synthesize ascorbate.

Also disclosed herein are methods of screening for a compound or composition for the treatment of hyperbilirubinemia. In some embodiments, the methods comprise contacting a hyperbilirubinemia liver organoid with the compound or composition, and detecting an improvement in the hyperbilirubinemia of the hyperbilirubinemia liver organoid.

Also disclosed herein are methods of treating a subject having a disease or disorder associated with bilirubin metabolism. In some embodiments, the methods comprise administering any one of the liver organoids or liver cells disclosed herein to the subject.

Also disclosed herein are the liver organoids or liver cells disclosed herein for use in the manufacture of a medicament for the treatment of a disease or disorder associated with bilirubin metabolism.

Also disclosed herein are the liver organoids or liver cells disclosed herein for use in the treatment of a disease or disorder associated with bilirubin metabolism in a subject in need thereof.

Exemplary embodiments of the present disclosure are provided in the following numbered embodiments:

1. A method of maturing a fetal-like liver organoid, comprising contacting a fetal-like liver organoid with a low/first concentration of bilirubin, thereby maturing the fetal-like liver organoid to a mature liver organoid.

2. The method of embodiment 1, wherein the low/first concentration of bilirubin is, is about, is less than, or is less than about: 0.1, 0.2, 0.3, 0.4, 0.5, 0.6, 0.7, 0.8, 0.9, 1, 1.25, 1.5, 1.75, 2.0, 2.25, 2.5, 2.75 or 3.0 mg/L, or any concentration within a range defined by any two of the aforementioned concentrations, for example, 0.1 to 3 mg/L, 0.5 to 2.0 mg/L, 0.5 to 1.5 mg/L, 0.3 to 2.5 mg/L, or 0.5 to 1.75 mg/L; or, 0.1, 0.2, 0.3, 0.4, 0.5, 0.6, 0.7, 0.8, 0.9, or 1 mg/L, or any concentration within a range defined by any two of the aforementioned concentrations, for example, 0.1 to 1 mg/L, 0.1 to 0.5 mg/L, 0.5 to 1 mg/L, 0.3 to 0.7 mg/L, or 0.4 to 0.6 mg/L.

3. The method of any preceding embodiment, wherein the mature liver organoid exhibits luminal projections that resemble bile canaliculi.

4. The method of any preceding embodiment, wherein the mature liver organoid expresses reduced levels of AFP, CDX2, NANOG, or any combination thereof, relative to the fetal-like liver organoid.

5. The method of any preceding embodiment, wherein the mature liver organoid expresses increased levels of ALB, SLC4A2, or HO-1, or any combination thereof, relative to the fetal-like liver organoid.

6. The method of any preceding embodiment, wherein the mature liver organoid expresses CYP2E1, CYP7A1, PROX1, MRP3, MRP3, or OATP2, or any combination thereof.

7. The method of any preceding embodiment, wherein the mature liver organoid exhibits CYP3A4 and CYP1A2 activity.

8. A method of producing a hyperbilirubinemia liver organoid, comprising contacting a liver organoid with a high/second concentration of bilirubin, thereby forming the hyperbilirubinemia liver organoid.

9. The method of embodiment 8, wherein the high/second concentration of bilirubin is, is about, is more than, or is more than about: 2, 3, 4, 5, 6, 7, 8, 9, 10, 11, 12, 13, 14, 15, 16, 17, 18, 19, or 20 mg/L, or any concentration within a range defined by any two of the aforementioned concentrations, for example, 2 to 20 mg/L, 2 to 10 mg/L, 10 to 20 mg/L, 5 to 15 mg/L, or 8 to 12 mg/L, or, 4, 5, 6, 7, 8, 9, 10, 11, 12, 13, 14, 15, 16, 17, 18, 19, or 20 mg/L, or any concentration within a range defined by any two of the aforementioned concentrations, for example, 4 to 20 mg/L, 2 to 10 mg/L, 10 to 20 mg/L, 5 to 15 mg/L, or 8 to 12 mg/L.

10. The method of embodiment 8 or 9, wherein the hyperbilirubinemia liver organoid expresses elevated levels of UGT1A1 or NRF2, or both, relative to a liver organoid not treated with a high/second concentration of bilirubin.

11. The method of any one of embodiments 8-10, further comprising contacting the hyperbilirubinemia liver organoid with ketoconazole or mifepristone, or both, to reduce the hyperbilirubinemia of the hyperbilirubinemia liver organoid.

12. A method of treating hyperbilirubinemia in a subject in need thereof, comprising administering ketoconazole or mifepristone, or both, to the subject in need thereof.

13. The mature liver organoid of any one of embodiments 1-7.

14. The hyperbilirubinemia liver organoid of any one of embodiments 8-11.

15. A liver organoid comprising a functional L-gulonolactone oxidase (GULO) gene.

16. The liver organoid of embodiment 15, wherein the functional GULO gene is a murine GULO (mGULO) gene.

17. A liver cell comprising a functional GULO gene.

18. The liver cell of embodiment 17, wherein the functional GULO gene is an mGULO gene.

19. A method of increasing bilirubin conjugation and metabolism in a liver cell, comprising expressing a functional GULO gene in the liver cell.

20. A method of screening for a compound or composition for the treatment of hyperbilirubinemia, comprising:
contacting a hyperbilirubinemia liver organoid with the compound or composition; and
detecting an improvement in the hyperbilirubinemia of the hyperbilirubinemia liver organoid.

21. The method of embodiment 20, wherein the hyperbilirubinemia liver organoid is the hyperbilirubinemia liver organoid of embodiment 14, wherein the hyperbilirubinemia liver organoid optionally comprises a functional GULO gene.

22. The method of embodiment 20 or 21, wherein detecting the improvement comprises detecting an increase in expression of UGT1A1 or NRF2, or both, relative to an untreated hyperbilirubinemia liver organoid.

23. The method of any one of embodiments 20-22, wherein detecting an improvement comprises detecting a relative increase in conjugated bilirubin to unconjugated bilirubin relative to an untreated hyperbilirubinemia liver organoid.

Further exemplary embodiments of the present disclosure are provided in the following numbered embodiments:

1. A method of maturing a fetal-like liver organoid, comprising contacting a fetal-like liver organoid with a low/first concentration of bilirubin, thereby maturing the fetal-like liver organoid to a mature liver organoid.

2. The method of embodiment 1, wherein the low/first concentration of bilirubin is a human fetal physiological concentration of bilirubin.

3. The method of embodiment 1 or 2, wherein the low/first concentration of bilirubin is, is about, is less than, or is less than about:
0.1, 0.2, 0.3, 0.4, 0.5, 0.6, 0.7, 0.8, 0.9, 1, 1.25, 1.5, 1.75, 2.0, 2.25, 2.5, 2.75 or 3.0 mg/L, or any concentration within a range defined by any two of the aforementioned concentrations, for example, 0.1 to 3 mg/L, 0.5 to 2.0 mg/L, 0.5 to 1.5 mg/L, 0.3 to 2.5 mg/L, or 0.5 to 1.75 mg/L; or
0.1, 0.2, 0.3, 0.4, 0.5, 0.6, 0.7, 0.8, 0.9, or 1 mg/L, or any concentration within a range defined by any two of the aforementioned concentrations, for example, 0.1 to 1 mg/L, 0.1 to 0.5 mg/L, 0.5 to 1 mg/L, 0.3 to 0.7 mg/L, or 0.4 to 0.6 mg/L.

4. The method of any one of the preceding embodiments, wherein the mature liver organoid exhibits luminal projections that resemble bile canaliculi.

5. The method of any one of the preceding embodiments, wherein the mature liver organoid expresses reduced levels of AFP, CDX2, NANOG, or any combination thereof, relative to the fetal-like liver organoid.

6. The method of any one of the preceding embodiments, wherein the mature liver organoid expresses increased levels of ALB, SLC4A2, or HO-1, or any combination thereof, relative to the fetal-like liver organoid.

7. The method of any one of the preceding embodiments, wherein the mature liver organoid expresses CYP2E1, CYP7A1, PROX1, MRP3, MRP3, or OATP2, or any combination thereof.

8 The method of any one of the preceding embodiments, wherein the mature liver organoid exhibits increased CYP3A4 and CYP1A2 activity relative to the fetal-like liver organoid.

9. The method of any of the preceding embodiments, wherein the mature liver organoid comprises a functional GULO protein and/or a gene or mRNA, or both, that encodes for the functional GULO protein, whereby the mature liver organoid is able to synthesize ascorbate.

10. The method of embodiment 9, wherein the functional GULO protein is murine GULO (mGULO).

11. The method of embodiment 9 or 10, wherein the gene that encodes for the functional GULO protein is conditionally expressed, optionally using a tetracycline inducible system.

12. The method of any one of embodiments 9-11, wherein the mature liver organoid is engineered with the gene that encodes for the functional GULO protein using CRISPR.

13. The method of any one of embodiments 9-12, wherein the gene or mRNA, or both, that encodes for the functional GULO protein is introduced to the mature liver organoid by transfection.

14. The method of any one of embodiments 9-13, wherein the mature liver organoid comprising the functional GULO protein expresses increased levels of NRF2 relative to the fetal-like liver organoid or a mature liver organoid that does not comprise the functional GULO protein.

15. The method of any one of embodiments 9-14, wherein the mature liver organoid comprising the functional GULO protein expresses reduced levels of IL1B, IL6, or TNFa, or any combination thereof, relative to the fetal-like liver organoid or a mature liver organoid that does not comprise the functional GULO protein, optionally when cultured in ascorbate-depleted medium or in the absence of ascorbate.

16. The method of any one of embodiments 9-15, wherein the mature liver organoid comprising the functional GULO protein exhibits reduced caspase-3 activity relative to the fetal-like liver organoid or a mature liver organoid that does not comprise the functional GULO protein, optionally when cultured in ascorbate-depleted medium.

17. The method of any one of embodiments 9-16, wherein the mature liver organoid comprising the functional GULO protein expresses increased levels of ALB relative to the fetal-like liver organoid or a mature liver organoid that does not comprise the functional GULO protein.

18. The method of any one of embodiments 9-17, wherein the mature liver organoid comprising the functional GULO protein resembles periportal liver tissue and expresses periportal liver markers.

19. The method of embodiment 18, wherein the periportal markers comprise FAH, ALB, PAH, CPS1, HGD, or any combination thereof.

20. The method of any one of embodiments 9-19, wherein the mature liver organoid comprising the functional GULO protein exhibits increased CYP3A4 and CYP1A2 activity relative to the fetal-like liver organoid or a mature liver organoid that does not comprise the functional GULO protein.

21 The method of any one of embodiments 9-20, wherein the mature liver organoid comprising the functional GULO protein exhibits increased bilirubin conjugation activity relative to the fetal-like liver organoid or a mature liver organoid that does not comprise the functional GULO protein.

22. The method of any one of embodiments 9-21, wherein the mature liver organoid comprising the functional GULO protein exhibits increased viability in culture relative to the fetal-like liver organoid or a mature liver organoid that does not comprise the functional GULO protein.

23 The method of any one of the preceding embodiments, wherein the fetal-like liver organoid is contacted with the low/first concentration of bilirubin in a hepatocyte culture medium.

24. The method of embodiment 23, wherein the hepatocyte culture medium comprises hepatocyte growth factor, oncostatin M, dexamethasone, or any combination thereof.

25 The method of any one of the preceding embodiments, wherein the mature liver organoid is human.

26. The method of any one of the preceding embodiments, wherein the mature liver organoid comprises an inactive UGT1A1 gene, wherein the mature liver organoid is a model for Crigler-Najjar Syndrome.

27. The method of any one of the preceding embodiments, wherein the fetal-like liver organoid has been differentiated from pluripotent stem cells, optionally embryonic stem cells or induced pluripotent stem cells.

28. The method of embodiment 27, wherein the pluripotent stem cells comprise a functional GULO protein and/or a gene or mRNA, or both, that encodes for the functional GULO protein, whereby the pluripotent stem cells are able to synthesize ascorbate.

29. The method of any one of the preceding embodiments, wherein the fetal-like liver organoid has been made according to a method comprising:
a) contacting definitive endoderm cells (DE) with an FGF signaling pathway activator and a Wnt signaling pathway activator for a first period of time;
b) contacting the cells of step a) with the FGF signaling pathway activator, the Wnt signaling pathway activator, and a retinoic acid (RA) signaling pathway activator for a second period of time, thereby differentiating the DE to posterior foregut cells; and
c) embedding the posterior foregut cells in a basement membrane matrix and culturing the posterior foregut spheroids for a third period of time to differentiate the posterior foregut cells to the fetal-like liver organoid.

30. A method of producing a hyperbilirubinemia liver organoid, comprising contacting a liver organoid with a high/second concentration of bilirubin, thereby forming the hyperbilirubinemia liver organoid.

31. The method of embodiment 30, wherein the high/second concentration of bilirubin is, is about, is more than, or is more than about:
2, 3, 4, 5, 6, 7, 8, 9, 10, 11, 12, 13, 14, 15, 16, 17, 18, 19, or 20 mg/L, or any concentration within a range defined by any two of the aforementioned concentrations, for example, 2 to 20 mg/L, 2 to 10 mg/L, 10 to 20 mg/L, 5 to 15 mg/L, or 8 to 12 mg/L, or
4, 5, 6, 7, 8, 9, 10, 11, 12, 13, 14, 15, 16, 17, 18, 19, or 20 mg/L, or any concentration within a range defined by any two of the aforementioned concentrations, for example, 4 to 20 mg/L, 2 to 10 mg/L, 10 to 20 mg/L, 5 to 15 mg/L, or 8 to 12 mg/L.

32. The method of embodiment 30 or 31, wherein the hyperbilirubinemia liver organoid expresses elevated levels of UGT1A1 or NRF2, or both, relative to a liver organoid not treated with a high/second concentration of bilirubin.

33. The method of any one of embodiments 30-32, further comprising contacting the hyperbilirubinemia liver organoid with a glucocorticoid antagonist to reduce the hyperbilirubinemia of the hyperbilirubinemia liver organoid.

34. The method of any one of embodiments 30-33, wherein contacting the hyperbilirubinemia liver organoid with the glucocorticoid antagonist increases expression of UGT1A1 and NRF2, and increases bilirubin conjugation activity in the hyperbilirubinemia liver organoid.

35. The method of embodiment 34, wherein the glucocorticoid antagonist is ketoconazole, mifepristone, or both, or is ketoconazole, mifepristone, metyrapone, aminoglutethimide, or any combination thereof.

36. The method of any one of embodiments 30-35, wherein the hyperbilirubinemia liver organoid comprises a functional GULO protein and/or a gene or mRNA, or both, that encodes for the functional GULO protein, wherein the hyperbilirubinemia liver organoid is able to synthesize ascorbate.

37. The method of embodiment 36, wherein the functional GULO protein is mGULO.

38 The method of embodiment 36 or 37, wherein the gene that encodes for the functional GULO protein is conditionally expressed, optionally using a tetracycline inducible system.

39. The method of any one of embodiments 30-38, wherein the hyperbilirubinemia liver organoid comprises an inactive UGT1A1 gene, wherein the hyperbilirubinemia liver organoid is a model for Crigler-Najjar Syndrome.

40. A method of treating hyperbilirubinemia in a subject in need thereof, comprising administering a glucocorticoid antagonist to the subject in need thereof, optionally wherein the glucocorticoid antagonist is ketoconazole, mifepristone, or both, or is ketoconazole, mifepristone, metyrapone, aminoglutethimide, or any combination thereof.

41 A mature liver organoid made by the method of any one of embodiments 1-29.

42. A hyperbilirubinemia liver organoid made by the method of any one of embodiments 30-39.

43. A liver organoid comprising a functional GULO protein and/or a gene or mRNA, or both, that encodes for the functional GULO protein, whereby the liver organoid is able to synthesize ascorbate.

44. The liver organoid of embodiment 43, wherein the functional GULO protein is mGULO.

45. The liver organoid of embodiment 43 or 44, wherein the gene that encodes for the functional GULO protein is conditionally expressed, optionally using a tetracycline inducible system.

46. The liver organoid of any one of embodiments 43-45, wherein the liver organoid is engineered with the gene that encodes for the functional GULO protein using CRISPR.

47. The liver organoid of any one of embodiments 43-46, wherein the gene or mRNA, or both, that encodes for the functional GULO protein is introduced to the mature liver organoid by transfection.

48. The liver organoid of any one of embodiments 43-47, wherein the liver organoid comprising the functional GULO protein expresses increased levels of NRF2 relative to a liver organoid that does not comprise the functional GULO protein.

49. The liver organoid of any one of embodiments 43-48, wherein the liver organoid comprising the functional GULO protein expresses reduced levels of IL1B, IL6, or TNFa, or any combination thereof, relative to a liver organoid that does not comprise the functional GULO protein, optionally when cultured in ascorbate-depleted medium.

50. The liver organoid of any one of embodiments 43-49, wherein the liver organoid comprising the functional GULO protein exhibits reduced caspase-3 activity relative to a liver organoid that does not comprise the functional GULO protein, optionally when cultured in ascorbate-depleted medium.

51. The liver organoid of any one of embodiments 43-50, wherein the liver organoid comprising the functional GULO protein expresses increased levels of ALB relative to a liver organoid that does not comprise the functional GULO protein.

52. The liver organoid of any one of embodiments 43-51, wherein the liver organoid comprising the functional GULO protein resembles periportal liver tissue and expresses a periportal liver marker.

53. The liver organoid of embodiment 52, wherein the periportal marker is selected from the group consisting of FAH, ALB, PAH, CPS1, HGD, and any combination thereof, optionally wherein the marker comprises FAH, ALB, PAH, CPS1, and HGD.

54. The liver organoid of any one of embodiments 43-53, wherein the liver organoid comprising the functional GULO protein exhibits increased CYP3A4 and CYP1A2 activity relative to a liver organoid that does not comprise the functional GULO protein.

55 The liver organoid of any one of embodiments 43-54, wherein the liver organoid comprising the functional GULO protein exhibits increased bilirubin conjugation activity relative to a liver organoid that does not comprise the functional GULO protein.

56. The liver organoid of any one of embodiments 43-55, wherein the liver organoid comprising the functional GULO protein exhibits increased viability in culture relative to a liver organoid that does not comprise the functional GULO protein.

57 The liver organoid of any one of embodiments 43-56, wherein the liver organoid is human.

58 The liver organoid of any one of embodiments 43-57, wherein the liver organoid comprises an inactive UGT1A1 gene, wherein the liver organoid is a model for Crigler-Najjar Syndrome.

59 The liver organoid of any one of embodiments 43-58, wherein the liver organoid has been differentiated from pluripotent stem cells, optionally embryonic stem cells or induced pluripotent stem cells.

60. The liver organoid of embodiment 59, wherein the pluripotent stem cells have been engineered with the functional GULO protein and/or a gene or mRNA, or both, that encodes for the functional GULO protein, whereby the pluripotent stem cells are able to synthesize ascorbate.

61. A liver cell comprising a functional GULO protein and/or a gene or mRNA, or both, that encodes for the functional GULO protein, whereby the liver cell is able to synthesize ascorbate.

62. The liver cell of embodiment 61, wherein the functional GULO protein is mGULO.

63. The liver cell of embodiment 61 or 62, wherein the gene that encodes for the functional GULO protein is conditionally expressed, optionally using a tetracycline inducible system.

64. The liver cell of any one of embodiments 61-63, wherein the liver cell is engineered with the gene that encodes for the functional GULO protein using CRISPR.

65. The liver cell of any one of embodiments 61-64, wherein the gene or mRNA, or both, that encodes for the functional GULO protein is introduced to the liver cell by transfection.

66. A method of increasing bilirubin conjugation and metabolism in a liver cell, comprising expressing a functional GULO protein and/or a gene or mRNA, or both, that encodes for the functional GULO protein, whereby the liver cell is able to synthesize ascorbate.

67. A method of screening for a compound or composition for the treatment of hyperbilirubinemia, comprising:
contacting a hyperbilirubinemia liver organoid with the compound or composition; and
detecting an improvement in the hyperbilirubinemia of the hyperbilirubinemia liver organoid.

68. The method of embodiment 67, wherein the hyperbilirubinemia liver organoid is the hyperbilirubinemia liver organoid of embodiment 42, wherein the hyperbilirubinemia liver organoid optionally comprises a functional GULO protein and/or a gene or mRNA, or both, that encodes for the functional GULO protein.

69. The method of embodiment 67 or 68, wherein detecting the improvement comprises detecting an increase in expression of UGT1A1 or NRF2, or both, relative to an untreated hyperbilirubinemia liver organoid.

70. The method of any one of embodiments 67-69, wherein detecting an improvement comprises detecting a relative increase in conjugated bilirubin to unconjugated bilirubin relative to an untreated hyperbilirubinemia liver organoid.

71 A method of treating a subject having a disease or disorder associated with bilirubin metabolism, comprising administering the liver organoid of any one of embodiments 43-60 or the liver cell of any one of embodiments 61-65 to the subject.

72. The liver organoid of any one of embodiments 43-60 or the liver cell of any one of embodiments 61-65 for use in the manufacture of a medicament for the treatment of a disease or disorder associated with bilirubin metabolism.

73. The liver organoid of any one of embodiments 43-60 or the liver cell of any one of embodiments 61-65 for the use in the treatment of a disease or disorder associated with bilirubin metabolism in a subject in need thereof.

74. The method of embodiment 71 or use of embodiment 72 or 73, wherein the disease or disorder associated with bilirubin metabolism is hyperbilirubinemia, jaundice, Crigler-Najjar syndrome, Gilbert's syndrome, Dubin-Johnson syndrome, or Rotor syndrome.

### BRIEF DESCRIPTION OF THE DRAWINGS

In addition to the features described herein, additional features and variations will be readily apparent from the following descriptions of the drawings and exemplary embodiments. It is to be understood that these drawings depict embodiments and are not intended to be limiting in scope.

FIG. 1A depicts an embodiment of a schematic for generation of HLOs and maturation with low dose bilirubin.

FIG. 1B depicts an embodiment of brightfield images of HLOs treated with low dose bilirubin (1 mg/L) compared to control, and luminal outline using ImageJ, where arrows indicate luminal projections that are similar to bile canaliculi found in human liver.

FIG. 1C depicts an embodiment of comparison of size and circularity of the lumen of the control and 1 mg/L bilirubin treated HLOs.

FIG. 1D depict an embodiment of RT-qPCR of immature and maturation marker genes (ALB, NANOG, SLC4A2, HO-1, AFP, and CDX2) for control organoids and organoids treated with 1 mg/L of bilirubin compared to a human liver sample.

FIG. 1E depict an embodiment of CYP3A4 and CYP1A2 activity assays in response to rifampicin and omeprazole in control and 1 mg/L bilirubin treated HLOs (RLU: relative light units; CTG: CellTiter-Glo assay).

FIGS. 1F-H depict embodiments of immunofluorescence of mature liver enzymes and transport proteins in 1 mg/L bilirubin treated liver organoids. FIG. 1F depicts detection of CYP2E1 and MRP3. FIG. 1G depicts detection of CYP7A1 and MRP1. FIG. 1H depicts detection of PROX1 and OATP2.

FIG. 2A depicts an embodiment of a brightfield image of HLOs with ascorbic acid depletion at day 15 compared to control.

FIG. 2B depicts an embodiment of an exemplary workflow for the generation of mGULO iPSCs.

FIG. 2C depicts an embodiment of a linear map of the synthetic mGULO-mCherry gene designed for the expression of mGULO under the doxycycline activated TetOn system in hiPSCs and vector map of the pAAVS1-Ndi-CRISPRi (Gen1) plasmid used to clone the mGULO gene into the hiPSCs.

FIG. 2D depicts an embodiment of a schematic for the generation of HLOs using iPSCs modified to express the TetOn mGULO gene.

FIG. 2E depicts an embodiment of brightfield and fluorescence images of mCherry expression in doxycycline (Dox) treated mGULO HLOs compared to control HLOs.

FIG. 2F depicts an embodiment of brightfield and fluorescence images of mCherry expression in ascorbate depleted mGULO HLOs with or without Dox treatment at day

18.

FIG. 2G depicts an embodiment of an ELISA for mGULO protein expression and cellular antioxidant concentration in mGULO HLOs treated with Dox (10 or 100 ng/mL) compared to control HLOs.

FIG. 2H depicts an embodiment of RT-qPCR of inflammatory and detoxification marker genes (NRF2, IL1B, IL6, and TNFa) in ascorbate depleted Dox treated mGULO HLOs compared to ascorbate depleted control or mGULO HLOs.

FIG. 2I depicts an embodiment of a caspase 3 activity assay for ascorbate depleted Dox treated mGULO HLOs compared to ascorbate depleted control or mGULO HLOs.

FIG. 2J depicts an embodiment of a heatmap from RNA-seq showing that Dox treated mGULO HLOs express periportal markers compared to control HLOs.

FIG. 2K depicts an embodiment of gene upregulation categorized by function, showing that periportal pathways are overrepresented in Dox treated mGULO HLOs.

FIG. 2L depicts an embodiment of brightfield images of Dox treated mGULO and control HLOs with or without 1 mg/L bilirubin treatment, and luminal outline using ImageJ, where arrows indicate luminal projections that are similar to bile canaliculi found in human liver.

FIG. 2M depicts an embodiment of comparison of size and circularity of the lumen of Dox treated mGULO HLOs or control HLOs with or without 1 mg/L bilirubin treatment.

FIG. 2N depicts an embodiment of quantification of albumin expression in Dox treated mGULO HLOs or control HLOs with or without 1 mg/L bilirubin treatment.

FIG. 2O depicts an embodiment of brightfield images of mGULO HLOs treated with bilirubin and Dox at varying concentrations (0, 10, 100, or 1000 ng/mL Dox).

FIG. 2P depicts an embodiment of CYP3A4 and CYP1A2 activity assays in response to rifampicin and omeprazole in control or Dox treated mGULO HLOs with 1 mg/L bilirubin.

FIG. 2Q depicts an embodiment of a UnaG assay showing loss of fluorescence indicating conjugation of bilirubin even in the presence of dark yellow bilirubin.

FIG. 2R depicts an embodiment of a UnaG assay for mGULO organoids treated with Dox compared to control.

FIG. 2S depicts an embodiment of quantification of the total percentage of viable organoids and organoids carrying conjugated bilirubin in Dox treated mGULO organoids compared to control.

FIG. 3A depicts an embodiment for a schematic for generation of HLOs and treatment with bilirubin at various concentrations.

FIG. 3B depicts an embodiment of brightfield images of HLOs treated with bilirubin (0-10 mg/L) after 1 and 4 days.

FIG. 3C depicts an embodiment of RT-qPCR of UGT1A1 and NRF2 genes for organoids treated with varying concentrations of bilirubin compared to untreated organoids.

FIG. 3D depicts an embodiment of a profile of a patient with Crigler-Najjar Syndrome (CNS) from whom CNS iPSCs were generated. DNA sequencing of the patient revealed a nonsense mutation c.858C>A (p.Cys280X) in the UGT1A1 gene.

FIG. 3E depicts an embodiment of fluorescence images showing that the CNS iPSCs derived from the patient with Crigler-Najjar Syndrome express canonical pluripotency markers Sox2 and Oct4.

FIG. 3F depicts an embodiment of brightfield images showing that the CNS iPSCs can be differentiated to definitive endoderm (DE) and liver organoids (hLO) according to standard protocols.

FIG. 3G depicts an embodiment of fluorescence images showing that liver organoids produced from the CNS iPSCs express the proliferation marker Ki67, liver-specific marker AFP, and epithelial marker ECAD.

FIG. 3H depicts an embodiment of brightfield images of CNS HLOs treated with bilirubin (10 mg/L) and control (0 mg/L bilirubin) after 1 and 4 days, showing that these HLOs suffer from bilirubin toxicity.

FIG. 3I depicts an embodiment of brightfield images of CNS HLOs and CNS HLOs that have been transfected with UGT1A1 mRNA at 10 days after treatment with bilirubin (10 mg/L).

FIG. 3J depicts an embodiment of a bilirubin assay measuring unconjugated (UCB) and conjugated (CB) bilirubin in the HLOs of FIG. 3I.

FIG. 3K depicts an embodiment of a bilirubin assay measuring unconjugated (UCB) and conjugated (CB) bilirubin in mGULO HLOs treated with 10 mg/L bilirubin and Dox (0, 10, 100, or 1000 ng/mL).

FIG. 4A depicts an embodiment of brightfield images of liver organoids treated with 10 mg/L bilirubin and glucocorticoid agonists hydrocortisone (HC; 1 or 5 μM) or dexamethasone (Dex; 1 or 5 μM).

FIG. 4B depicts an embodiment of a bilirubin assay measuring unconjugated and conjugated bilirubin in the liver organoids of FIG. 4A.

FIG. 4C depicts an embodiment of brightfield images of liver organoids treated with 10 mg/L bilirubin and glucocorticoid antagonists ketoconazole (KCZ; 1 or 5 μM) or mifepristone (Mif; 1 or 5 μM).

FIG. 4D depicts an embodiment of a bilirubin assay measuring unconjugated and conjugated bilirubin in the liver organoids of FIG. 4C.

FIG. 4E depicts an embodiment of RT-qPCR of UGT1A1 and NRF2 genes for organoids treated with 10 mg/L bilirubin compared to organoids treated with 10 mg/L bilirubin and hydrocortisone, dexamethasone, ketoconazole, or mifepristone.

FIG. 4F depicts an embodiment of a comparison of enriched pathways obtained from RNA sequencing between organoids treated with 10 mg/L bilirubin and 1 μM mifepristone compared to control and a GSEA plot comparing enriched ROS and xenobiotic metabolism.

FIG. 4G depicts an embodiment of a Venn diagram showing differentially expressed genes in ROS and xenobiotic metabolism.

FIG. 4H depicts an embodiment of ChIP-PCR and CHIP-qPCR for organoids treated with 10 mg/L bilirubin and either 1 μM mifepristone (Mife) or 1 μM dexamethasone (Dex).

FIG. 5A depicts an embodiment of a workflow for orthotopic transplantation of HLOs in rodents.

FIG. 5B depicts an embodiment of an albumin ELISA on blood serum collected from Gunn rats transplanted with mGULO HLOs or sham at different time points after transplantation.

FIG. 5C depicts an embodiment of a bilirubin assay on the Gunn rats of FIG. 5B after transplantation.

FIG. 5D depicts an embodiment of AST and ALT assays on the Gunn rats of FIG. 5B after transplantation.

### DETAILED DESCRIPTION

The incidence of liver diseases has increased at an accelerated pace. Neonatal hyperbilirubinemia (NH) is one condition that exacerbates the health of neonates. It affects 60% of all newborns and accounts for 114,000 annual deaths worldwide. The only treatment for NH is 12 hours of phototherapy or exchange transfusion, but they cause other complications. Therefore, efficient and scalable model systems for these liver diseases have now become a necessity to understand the molecular mechanism behind them and develop potential therapies.

There are currently two main model organisms for modeling NH: the Gunn rats and UGT1A1 knockout mice. However, these models lack key human proteins (OATP family) and epigenetic control involved in bilirubin metabolism. Recent studies have revealed that many aspects of UGT1A1 regulation do not translate well across the species. The genetic regulation of the UGT1A1 gene, which is the rate limiting enzyme in bilirubin metabolism, is significantly different in humans. Gunn rats with UGT deficiency are quite hard to maintain and require special accommodations. In addition, UGT1 KO mouse models display lethal hyperbilirubinemia and only live up to a couple of weeks. Many treatments have been tested with these models, but most have failed. Thus, there is a critical need to develop a more commensurable model to understand the dynamics of bilirubin metabolism.

Herein, human liver organoids (HLOs) are used to model hyperbilirubinemia by treating them with varying concentrations of bilirubin. These HLOs can be derived from patient derived induced pluripotent stem cells (iPSCs), where the patient can be healthy or having a diseased condition, and are identical in genetic content to the respective patient. They express most liver markers that are expressed in the pre-natal stages of development. Furthermore, they are clonal and therefore reacts similarly to external stimuli and biochemical perturbations. These HLOs are highly scalable and tractable, allowing screening approaches to test a vast array of drugs and small molecules.

Breeding model organisms such as mice and rats takes months of work and planning, and the chance of getting the desired genotype is relatively low. Furthermore, model organisms show high variations in responses to biochemical perturbations over generations. These rodents also run the risk of losing the desired genotype when bred over long periods of time, and also require complex training and procedures to model diseases and evaluate the efficacy of treatments. Conversely, HLOs are easy to work with and have very low variation across batches. Large batches of HLOs can be generated within a couple of weeks. Leveraging these qualities, several drugs were tested within a short span of time to identify a critical pathway that is involved in bilirubin metabolism. Therefore, liver organoids are a useful model for studying diseases and disorders associated with dysfunctional bilirubin metabolism, such as jaundice, Crigler-Najjar syndrome, Gilbert's syndrome, Dubin-Johnson syndrome, or Rotor syndrome.

Although abnormal metabolism of bilirubin results in disease, bilirubin is an important metabolite during early fetal development and acts as a metabolic hormone to play an antioxidant role in adults. HLO models prepared by previous methods, which did not involve the use of bilirubin, resemble immature tissue and express fetal and intestinal markers. Accordingly, as also disclosed herein, maturation of HLOs were induced by using low doses (mimicking normal physiological levels) of bilirubin. Modulating the glucocorticoid receptor pathway in these HLOs, such as with treatment with mifepristone and ketoconazole, improved conjugation and metabolism of bilirubin.

Vitamin C is also necessary for proper development of the fetus and involved in the formation of the periportal zone of the liver. L-gulonolactone oxidase (GULO) is a naturally occurring enzyme that synthesizes vitamin C, but this enzyme is non-functional in human and some other animals such as Guinea pigs, necessitating exogenous vitamin C supplementation (typically through the diet). As shown in Guinea pig animal models, vitamin C deficiency causes significant metabolic disorders.

Compared to model organisms, genetic modifications are much easier in iPSC cell lines and they can be maintained easily over longer periods before differentiation into organoids. Taking advantage of this, iPSC-derived organoids expressing a functional L-gulonolactone oxidase (GULO), such as murine GULO (mGULO), were generated. When the iPSCs and organoids are human in origin, the expression of the functional L-gulonolactone allows for ascorbate synthesis, which is normally inactive in humans. These mGULO organoids exhibited increased efficiency in conjugating bilirubin and exhibited improved viability when treated with bilirubin. The production of ascorbate in mGULO organoids reduces oxidative stress in the organoids and drives expression of NRF2, which is a master regulator of cellular detoxicification pathways and in turn promotes expression of UGT1A1, which catalyzes bilirubin conjugation. These mGULO organoids are otherwise genetically identical to the patients from which they are derived, and encompass the aspects of human bilirubin metabolism. Accordingly, these organoids can be used as model systems for elucidating the mechanistic development of NH and developing therapeutic treatments thereto.

### Terms

In the following detailed description, reference is made to the accompanying drawings, which form a part hereof. In the drawings, similar symbols typically identify similar components, unless context dictates otherwise. The illustrative embodiments described in the detailed description, drawings, and claims are not meant to be limiting. Other embodiments may be utilized, and other changes may be made, without departing from the spirit or scope of the subject matter presented herein. It will be readily understood that the aspects of the present disclosure, as generally described herein, and illustrated in the Figures, can be arranged, substituted, combined, separated, and designed in a wide variety of different configurations, all of which are explicitly contemplated herein.

Unless defined otherwise, technical and scientific terms used herein have the same meaning as commonly understood when read in light of the instant disclosure by one of ordinary skill in the art to which the present disclosure belongs. For purposes of the present disclosure, the following terms are explained below.

The disclosure herein uses affirmative language to describe the numerous embodiments. The disclosure also includes embodiments in which subject matter is excluded, in full or in part, such as substances or materials, method steps and conditions, protocols, or procedures.

The articles “a” and “an” are used herein to refer to one or to more than one (for example, at least one) of the grammatical object of the article. By way of example, “an element” means one element or more than one element.

By “about” is meant a quantity, level, value, number, frequency, percentage, dimension, size, amount, weight or length that varies by as much as 10% to a reference quantity, level, value, number, frequency, percentage, dimension, size, amount, weight or length.

Throughout this specification, unless the context requires otherwise, the words “comprise,” “comprises,” and “comprising” will be understood to imply the inclusion of a stated step or element or group of steps or elements but not the exclusion of any other step or element or group of steps or elements. By “consisting of” is meant including, and limited to, whatever follows the phrase “consisting of.” Thus, the phrase “consisting of” indicates that the listed elements are required or mandatory, and that no other elements may be present. By “consisting essentially of” is meant including any elements listed after the phrase, and limited to other elements that do not interfere with or contribute to the activity or action specified in the disclosure for the listed elements. Thus, the phrase “consisting essentially of” indicates that the listed elements are required or mandatory, but that other elements are optional and may or may not be present depending upon whether or not they materially affect the activity or action of the listed elements.

The terms “individual”, “subject”, or “patient” as used herein have their plain and ordinary meaning as understood in light of the specification, and mean a human or a non-human mammal, e.g., a dog, a cat, a mouse, a rat, a cow, a sheep, a pig, a goat, a non-human primate, or a bird, e.g., a chicken, as well as any other vertebrate or invertebrate. The term “mammal” is used in its usual biological sense. Thus, it specifically includes, but is not limited to, primates, including simians (chimpanzees, apes, monkeys) and humans, cattle, horses, sheep, goats, swine, rabbits, dogs, cats, rodents, rats, mice, guinea pigs, or the like.

The terms “effective amount” or “effective dose” as used herein have their plain and ordinary meaning as understood in light of the specification, and refer to that amount of a recited composition or compound that results in an observable effect. Actual dosage levels of active ingredients in an active composition of the presently disclosed subject matter can be varied so as to administer an amount of the active composition or compound that is effective to achieve the desired response for a particular subject and/or application. The selected dosage level will depend upon a variety of factors including, but not limited to, the activity of the composition, formulation, route of administration, combination with other drugs or treatments, severity of the condition being treated, and the physical condition and prior medical history of the subject being treated. In some embodiments, a minimal dose is administered, and dose is escalated in the absence of dose-limiting toxicity to a minimally effective amount. Determination and adjustment of an effective dose, as well as evaluation of when and how to make such adjustments, are contemplated herein.

The terms “function” and “functional” as used herein have their plain and ordinary meaning as understood in light of the specification, and refer to a biological, enzymatic, or therapeutic function.

The term “inhibit” as used herein has its plain and ordinary meaning as understood in light of the specification, and may refer to the reduction or prevention of a biological activity. The reduction can be by a percentage that is, is about, is at least, is at least about, is not more than, or is not more than about, 10%, 20%, 30%, 40%, 50%, 60%, 70%, 80%, 90%, or 100%, or an amount that is within a range defined by any two of the aforementioned values. As used herein, the term “delay” has its plain and ordinary meaning as understood in light of the specification, and refers to a slowing, postponement, or deferment of a biological event, to a time which is later than would otherwise be expected. The delay can be a delay of a percentage that is, is about, is at least, is at least about, is not more than, or is not more than about, 0%, 10%, 20%, 30%, 40%, 50%, 60%, 70%, 80%, 90%, 100%, or an amount within a range defined by any two of the aforementioned values. The terms inhibit and delay may not necessarily indicate a 100% inhibition or delay. A partial inhibition or delay may be realized.

As used herein, the term “isolated” has its plain and ordinary meaning as understood in light of the specification, and refers to a substance and/or entity that has been (1) separated from at least some of the components with which it was associated when initially produced (whether in nature and/or in an experimental setting), and/or (2) produced, prepared, and/or manufactured by the hand of man. Isolated substances and/or entities may be separated from equal to, about, at least, at least about, not more than, or not more than about, 10%, about 20%, about 30%, about 40%, about 50%, about 60%, about 70%, about 80%, about 90%, about 95%, about 98%, about 99%, substantially 100%, or 100% of the other components with which they were initially associated (or ranges including and/or spanning the aforementioned values). In some embodiments, isolated agents are, are about, are at least, are at least about, are not more than, or are not more than about 80%, about 85%, about 90%, about 91%, about 92%, about 93%, about 94%, about 95%, about 96%, about 97%, about 98%, about 99%, substantially 100%, or 100% pure (or ranges including and/or spanning the aforementioned values). As used herein, a substance that is “isolated” may be “pure” (e.g., substantially free of other components). As used herein, the term “isolated cell” may refer to a cell not contained in a multi-cellular organism or tissue.

As used herein, “in vivo” is given its plain and ordinary meaning as understood in light of the specification and refers to the performance of a method inside living organisms, usually animals, mammals, including humans, and plants, as opposed to a tissue extract or dead organism.

As used herein, “ex vivo” is given its plain and ordinary meaning as understood in light of the specification and refers to the performance of a method outside a living organism with little alteration of natural conditions.

As used herein, “in vitro” is given its plain and ordinary meaning as understood in light of the specification and refers to the performance of a method outside of biological conditions, e.g., in a petri dish or test tube.

The terms “nucleic acid” or “nucleic acid molecule” as used herein have their plain and ordinary meaning as understood in light of the specification, and refer to polynucleotides, such as deoxyribonucleic acid (DNA) or ribonucleic acid (RNA), oligonucleotides, those that appear in a cell naturally, fragments generated by the polymerase chain reaction (PCR), and fragments generated by any of ligation, scission, endonuclease action, and exonuclease action. Nucleic acid molecules can be composed of monomers that are naturally-occurring nucleotides (such as DNA and RNA), or analogs of naturally-occurring nucleotides (e.g., enantiomeric forms of naturally-occurring nucleotides), or a combination of both. Modified nucleotides can have alterations in sugar moieties and/or in pyrimidine or purine base moieties. Sugar modifications include, for example, replacement of one or more hydroxyl groups with halogens, alkyl groups, amines, and azido groups, or sugars can be functionalized as ethers or esters. Moreover, the entire sugar moiety can be replaced with sterically and electronically similar structures, such as aza-sugars and carbocyclic sugar analogs. Examples of modifications in a base moiety include alkylated purines and pyrimidines, acylated purines or pyrimidines, or other well-known heterocyclic substitutes. Nucleic acid monomers can be linked by phosphodiester bonds or analogs of such linkages. Analogs of phosphodiester linkages include phosphorothioate, phosphorodithioate, phosphoroselenoate, phosphorodiselenoate, phosphoroanilothioate, phosphoranilidate, or phosphoramidate. The term “nucleic acid molecule” also includes so-called “peptide nucleic acids,” which comprise naturally-occurring or modified nucleic acid bases attached to a polyamide backbone. Nucleic acids can be either single stranded or double stranded. “Oligonucleotide” can be used interchangeable with nucleic acid and can refer to either double stranded or single stranded DNA or RNA. A nucleic acid or nucleic acids can be contained in a nucleic acid vector or nucleic acid construct (e.g. plasmid, virus, retrovirus, lentivirus, bacteriophage, cosmid, fosmid, phagemid, bacterial artificial chromosome (BAC), yeast artificial chromosome (YAC), or human artificial chromosome (HAC)) that can be used for amplification and/or expression of the nucleic acid or nucleic acids in various biological systems. Typically, the vector or construct will also contain elements including but not limited to promoters, enhancers, terminators, inducers, ribosome binding sites, translation initiation sites, start codons, stop codons, polyadenylation signals, origins of replication, cloning sites, multiple cloning sites, restriction enzyme sites, epitopes, reporter genes, selection markers, antibiotic selection markers, targeting sequences, peptide purification tags, or accessory genes, or any combination thereof.

A nucleic acid or nucleic acid molecule can comprise one or more sequences encoding different peptides, polypeptides, or proteins. These one or more sequences can be joined in the same nucleic acid or nucleic acid molecule adjacently, or with extra nucleic acids in between, e.g. linkers, repeats or restriction enzyme sites, or any other sequence that is, is about, is at least, is at least about, is not more than, or is not more than about, 1, 2, 3, 4, 5, 6, 7, 8, 9, 10, 11, 12, 13, 14, 15, 16, 17, 18, 19, 20, 25, 30, 35, 40, 45, 50, 55, 60, 65, 70, 75, 80, 85, 90, 95, 100, 150, 200, or 300 bases long, or any length in a range defined by any two of the aforementioned lengths. The term “downstream” on a nucleic acid as used herein has its plain and ordinary meaning as understood in light of the specification and refers to a sequence being after the 3′-end of a previous sequence, on the strand containing the encoding sequence (sense strand) if the nucleic acid is double stranded. The term “upstream” on a nucleic acid as used herein has its plain and ordinary meaning as understood in light of the specification and refers to a sequence being before the 5′-end of a subsequent sequence, on the strand containing the encoding sequence (sense strand) if the nucleic acid is double stranded. The term “grouped” on a nucleic acid as used herein has its plain and ordinary meaning as understood in light of the specification and refers to two or more sequences that occur in proximity either directly or with extra nucleic acids in between, e.g. linkers, repeats, or restriction enzyme sites, or any other sequence that is, is about, is at least, is at least about, is not more than, or is not more than about, 1, 2, 3, 4, 5, 6, 7, 8, 9, 10, 11, 12, 13, 14, 15, 16, 17, 18, 19, 20, 25, 30, 35, 40, 45, 50, 55, 60, 65, 70, 75, 80, 85, 90, 95, 100, 150, 200, or 300 bases long, or any length in a range defined by any two of the aforementioned lengths, but generally not with a sequence in between that encodes for a functioning or catalytic polypeptide, protein, or protein domain.

The nucleic acids described herein comprise nucleobases. Primary, canonical, natural, or unmodified bases are adenine, cytosine, guanine, thymine, and uracil. Other nucleobases include but are not limited to purines, pyrimidines, modified nucleobases, 5-methylcytosine, pseudouridine, dihydrouridine, inosine, 7-methylguanosine, hypoxanthine, xanthine, 5,6-dihydrouracil, 5-hydroxymethylcytosine, 5-bromouracil, isoguanine, isocytosine, aminoallyl bases, dye-labeled bases, fluorescent bases, or biotin-labeled bases.

The terms “peptide”, “polypeptide”, and “protein” as used herein have their plain and ordinary meaning as understood in light of the specification and refer to macromolecules comprised of amino acids linked by peptide bonds. The numerous functions of peptides, polypeptides, and proteins are known in the art, and include but are not limited to enzymes, structure, transport, defense, hormones, or signaling. Peptides, polypeptides, and proteins are often, but not always, produced biologically by a ribosomal complex using a nucleic acid template, although chemical syntheses are also available. By manipulating the nucleic acid template, peptide, polypeptide, and protein mutations such as substitutions, deletions, truncations, additions, duplications, or fusions of more than one peptide, polypeptide, or protein can be performed. These fusions of more than one peptide, polypeptide, or protein can be joined in the same molecule adjacently, or with extra amino acids in between, e.g. linkers, repeats, epitopes, or tags, or any other sequence that is, is about, is at least, is at least about, is not more than, or is not more than about, 1, 2, 3, 4, 5, 6, 7, 8, 9, 10, 11, 12, 13, 14, 15, 16, 17, 18, 19, 20, 25, 30, 35, 40, 45, 50, 55, 60, 65, 70, 75, 80, 85, 90, 95, 100, 150, 200, or 300 bases long, or any length in a range defined by any two of the aforementioned lengths. The term “downstream” on a polypeptide as used herein has its plain and ordinary meaning as understood in light of the specification and refers to a sequence being after the C-terminus of a previous sequence. The term “upstream” on a polypeptide as used herein has its plain and ordinary meaning as understood in light of the specification and refers to a sequence being before the N-terminus of a subsequent sequence.

The term “purity” of any given substance, compound, or material as used herein has its plain and ordinary meaning as understood in light of the specification and refers to the actual abundance of the substance, compound, or material relative to the expected abundance. For example, the substance, compound, or material may be at least 80, 85, 90, 91, 92, 93, 94, 95, 96, 97, 98, 99, or 100% pure, including all decimals in between. Purity may be affected by unwanted impurities, including but not limited to nucleic acids, DNA, RNA, nucleotides, proteins, polypeptides, peptides, amino acids, lipids, cell membrane, cell debris, small molecules, degradation products, solvent, carrier, vehicle, or contaminants, or any combination thereof. In some embodiments, the substance, compound, or material is substantially free of host cell proteins, host cell nucleic acids, plasmid DNA, contaminating viruses, proteasomes, host cell culture components, process related components, mycoplasma, pyrogens, bacterial endotoxins, and adventitious agents. Purity can be measured using technologies including but not limited to electrophoresis, SDS-PAGE, capillary electrophoresis, PCR, rtPCR, qPCR, chromatography, liquid chromatography, gas chromatography, thin layer chromatography, enzyme-linked immunosorbent assay (ELISA), spectroscopy, UV-visible spectrometry, infrared spectrometry, mass spectrometry, nuclear magnetic resonance, gravimetry, or titration, or any combination thereof.

The term “yield” of any given substance, compound, or material as used herein has its plain and ordinary meaning as understood in light of the specification and refers to the actual overall amount of the substance, compound, or material relative to the expected overall amount. For example, the yield of the substance, compound, or material is, is about, is at least, is at least about, is not more than, or is not more than about, 80, 85, 90, 91, 92, 93, 94, 95, 96, 97, 98, 99, or 100% of the expected overall amount, including all decimals in between. Yield may be affected by the efficiency of a reaction or process, unwanted side reactions, degradation, quality of the input substances, compounds, or materials, or loss of the desired substance, compound, or material during any step of the production.

As used herein, “pharmaceutically acceptable” has its plain and ordinary meaning as understood in light of the specification and refers to carriers, excipients, and/or stabilizers that are nontoxic to the cell or mammal being exposed thereto at the dosages and concentrations employed or that have an acceptable level of toxicity. A “pharmaceutically acceptable” “diluent,” “excipient,” and/or “carrier” as used herein have their plain and ordinary meaning as understood in light of the specification and are intended to include any and all solvents, dispersion media, coatings, antibacterial and antifungal agents, isotonic and absorption delaying agents, and the like, compatible with administration to humans, cats, dogs, or other vertebrate hosts. Typically, a pharmaceutically acceptable diluent, excipient, and/or carrier is a diluent, excipient, and/or carrier approved by a regulatory agency of a Federal, a state government, or other regulatory agency, or listed in the U.S. Pharmacopeia or other generally recognized pharmacopeia for use in animals, including humans as well as non-human mammals, such as cats and dogs. The term diluent, excipient, and/or “carrier” can refer to a diluent, adjuvant, excipient, or vehicle with which the pharmaceutical composition is administered. Such pharmaceutical diluent, excipient, and/or carriers can be sterile liquids, such as water and oils, including those of petroleum, animal, vegetable or synthetic origin. Water, saline solutions and aqueous dextrose and glycerol solutions can be employed as liquid diluents, excipients, and/or carriers, particularly for injectable solutions. Suitable pharmaceutical diluents and/or excipients include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. A non-limiting example of a physiologically acceptable carrier is an aqueous pH buffered solution. The physiologically acceptable carrier may also comprise one or more of the following: antioxidants, such as ascorbic acid, low molecular weight (less than about 10 residues) polypeptides, proteins, such as serum albumin, gelatin, immunoglobulins, hydrophilic polymers such as polyvinylpyrrolidone, amino acids, carbohydrates such as glucose, mannose, or dextrins, chelating agents such as EDTA, sugar alcohols such as mannitol or sorbitol, salt-forming counterions such as sodium, and nonionic surfactants such as TWEEN®, polyethylene glycol (PEG), and PLURONICS®. The composition, if desired, can also contain minor amounts of wetting, bulking, emulsifying agents, or pH buffering agents. These compositions can take the form of solutions, suspensions, emulsion, sustained release formulations and the like. The formulation should suit the mode of administration.

Cryoprotectants are cell composition additives to improve efficiency and yield of low temperature cryopreservation by preventing formation of large ice crystals. Cryoprotectants include but are not limited to DMSO, ethylene glycol, glycerol, propylene glycol, trehalose, formamide, methyl-formamide, dimethyl-formamide, glycerol 3-phosphate, proline, sorbitol, diethyl glycol, sucrose, triethylene glycol, polyvinyl alcohol, polyethylene glycol, or hydroxyethyl starch. Cryoprotectants can be used as part of a cryopreservation medium, which include other components such as nutrients (e.g. albumin, serum, bovine serum, fetal calf serum [FCS]) to enhance post-thawing survivability of the cells. In these cryopreservation media, at least one cryoprotectant may be found at a concentration that is, is about, is at least, is at least about, is not more than, or is not more than about, 0.01%, 0.05%, 0.1%, 0.5%, 1%, 2%, 3%, 4%, 5%, 6%, 7%, 8%, 9%, 10%, 11%, 12%, 13%, 14%, 15%, 16%, 17%, 18%, 19%, 20%, 30%, 40%, 50%, 60%, 70%, 80%, or 90%, or any percentage within a range defined by any two of the aforementioned numbers.

Additional excipients with desirable properties include but are not limited to preservatives, adjuvants, stabilizers, solvents, buffers, diluents, solubilizing agents, detergents, surfactants, chelating agents, antioxidants, alcohols, ketones, aldehydes, ethylenediaminetetraacetic acid (EDTA), citric acid, salts, sodium chloride, sodium bicarbonate, sodium phosphate, sodium borate, sodium citrate, potassium chloride, potassium phosphate, magnesium sulfate sugars, dextrose, fructose, mannose, lactose, galactose, sucrose, sorbitol, cellulose, serum, amino acids, polysorbate 20, polysorbate 80, sodium deoxycholate, sodium taurodeoxycholate, magnesium stearate, octylphenol ethoxylate, benzethonium chloride, thimerosal, gelatin, esters, ethers, 2-phenoxyethanol, urea, or vitamins, or any combination thereof. Some excipients may be in residual amounts or contaminants from the process of manufacturing, including but not limited to serum, albumin, ovalbumin, antibiotics, inactivating agents, formaldehyde, glutaraldehyde, β-propiolactone, gelatin, cell debris, nucleic acids, peptides, amino acids, or growth medium components or any combination thereof. The amount of the excipient may be found in composition at a percentage that is, is about, is at least, is at least about, is not more than, or is not more than about, 0%, 0.1%, 0.2%, 0.3%, 0.4%, 0.5%, 0.6%, 0.7%, 0.8%, 0.9%, 1%, 2%, 3%, 4%, 5%, 6%, 7%, 8%, 9%, 10%, 20%, 30%, 40%, 50%, 60%, 70%, 80%, 90%, 95%, 100% w/w or any percentage by weight in a range defined by any two of the aforementioned numbers.

The term “pharmaceutically acceptable salts” has its plain and ordinary meaning as understood in light of the specification and includes relatively non-toxic, inorganic and organic acid, or base addition salts of compositions or excipients, including without limitation, analgesic agents, therapeutic agents, other materials, and the like. Examples of pharmaceutically acceptable salts include those derived from mineral acids, such as hydrochloric acid and sulfuric acid, and those derived from organic acids, such as ethanesulfonic acid, benzenesulfonic acid, p-toluenesulfonic acid, and the like. Examples of suitable inorganic bases for the formation of salts include the hydroxides, carbonates, and bicarbonates of ammonia, sodium, lithium, potassium, calcium, magnesium, aluminum, zinc, and the like. Salts may also be formed with suitable organic bases, including those that are non-toxic and strong enough to form such salts. For example, the class of such organic bases may include but are not limited to mono-, di-, and trialkylamines, including methylamine, dimethylamine, and triethylamine; mono-, di-, or trihydroxyalkylamines including mono-, di-, and triethanolamine; amino acids, including glycine, arginine and lysine; guanidine; N-methylglucosamine; N-methylglucamine; L-glutamine; N-methylpiperazine; morpholine; ethylenediamine; N-benzylphenethylamine; trihydroxymethyl aminoethane.

Proper formulation is dependent upon the route of administration chosen. Techniques for formulation and administration of the compounds described herein are known to those skilled in the art. Multiple techniques of administering a compound exist in the art including, but not limited to, enteral, oral, rectal, topical, sublingual, buccal, intraaural, epidural, epicutaneous, aerosol, parenteral delivery, including intramuscular, subcutaneous, intra-arterial, intravenous, intraportal, intra-articular, intradermal, peritoneal, intramedullary injections, intrathecal, direct intraventricular, intraperitoneal, intranasal or intraocular injections. Pharmaceutical compositions will generally be tailored to the specific intended route of administration.

As used herein, a “carrier” has its plain and ordinary meaning as understood in light of the specification and refers to a compound, particle, solid, semi-solid, liquid, or diluent that facilitates the passage, delivery and/or incorporation of a compound to cells, tissues and/or bodily organs.

As used herein, a “diluent” has its plain and ordinary meaning as understood in light of the specification and refers to an ingredient in a pharmaceutical composition that lacks pharmacological activity but may be pharmaceutically necessary or desirable. For example, a diluent may be used to increase the bulk of a potent drug whose mass is too small for manufacture and/or administration. It may also be a liquid for the dissolution of a drug to be administered by injection, ingestion or inhalation. A common form of diluent in the art is a buffered aqueous solution such as, without limitation, phosphate buffered saline that mimics the composition of human blood.

The term “basement membrane matrix” or “extracellular matrix” as used herein has its plain and ordinary meaning in light of the specification and refers to any biological or synthetic compound, substance, or composition that enhances cell attachment and/or growth. Any extracellular matrix, as well as any mimetic or derivative thereof, known in the art can be used for the methods disclosed herein. Some examples of extracellular matrices, or mimetics or derivative thereof, include but are not limited to cell-based feeder layers, polymers, proteins, polypeptides, nucleic acids, sugars, lipids, poly-lysine, poly-ornithine, collagen, gelatin, fibronectin, vitronectin, laminin, elastin, tenascin, heparan sulfate, entactin, nidogen, osteopontin, basement membrane, Matrigel, hydrogel, PEI, WGA, or hyaluronic acid, or any combination thereof.

The microscopic architecture of the liver is made up of polygonal structures called “hepatic lobules”. Classically, these lobules take on a hexagonal structure, although other geometric shapes are observed depending on tissue specification. Each lobule unit comprises plates or layers of hepatocytes surrounding an internal central vein and encapsulated by bundles of vessels called portal triads, which are made up of a portal vein, hepatic artery, and bile duct. Hepatic activity occurs as blood flows from the portal triads at the periphery, across the hepatocytes, and into the central vein to return to the circulatory system. Due to the asymmetric organization of these lobules, the layers of hepatocytes are divided into three zones. Cells in the “periportal zone” (zone 1) are closest to the portal triad and receive the most oxygenated blood, the pericentral zone (zone 3) are closest to the central vein and therefore receive the least amount of oxygenated blood, and the transition zone (zone 2) is in between zone 1 and 3. Due to this separation, each zone of hepatocytes exhibit differing activities. For example, zone 1 hepatocytes are involved in oxidative liver functions such as gluconeogenesis and oxidative metabolism of fatty acids, whereas zone 3 hepatocytes are involved in glycolysis, lipogenesis, and cytochrome P450-mediated detoxification. In some embodiments, the liver organoids disclosed herein exhibit a periportal-like identity resembling the tissue found in the periportal zone of liver lobules, including the functional and cellular marker characteristics of the periportal zone.

The term “bilirubin” as used herein has its plain and ordinary meaning as understood in light of the specification and refers to the naturally occurring metabolite created by normal catabolic degradation of heme. Bilirubin arises from the catalysis of biliverdin by biliverdin reductase. In the liver, bilirubin is conjugated with glucuronic acid by a family of enzymes called UDT-glucuronosyltransferases (UGTs). This conjugation renders bilirubin water soluble, enabling it to be carried in bile to the small intestine and colon, whereby it is further metabolized to waste products. Dysfunctional bilirubin metabolism, particularly due to abnormal function of UGTs preventing conjugation of bilirubin, leads to accumulation of bilirubin and is associated with various diseases characterized by hyperbilirubinemia. Notably, however, while excessive bilirubin is detrimental, bilirubin also has antioxidant capabilities and therefore may have beneficial effects in reducing oxidative damage in cells.

The term “L-gulonolactone oxidase” and “GULO” as used herein has its plain and ordinary meaning as understood in light of the specification and refers to the enzyme that catalyzes L-gulonolactone to produce L-xylo-hex-3-gulonolactone and hydrogen peroxide. The L-xylo-hex-3-gulonolactone then spontaneously converts to ascorbate (vitamin C). Accordingly, this enzyme is involved in the biosynthesis of vitamin C, which is an essential nutrient that is involved in many biological functions such as use as a cofactor for several important enzymes and as an antioxidant. Notably, humans, as well as other haplorrhine primates, certain species of bats, and Guinea pigs have evolved to harbor a non-functional GULO gene. Therefore, these organisms are unable to synthesize ascorbate and require vitamin C intake from diet or supplementation, where a deficiency of vitamin C can lead to scurvy. As applied to the disclosure herein, a “functional GULO protein” is a GULO protein that has L-gulonolactone catalytic activity to result in the production of ascorbate. Conversely, an “inactive” GULO protein or “non-functional” GULO protein is one that does not have the catalytic activity to produce ascorbate. Humans and cells that are derived from humans comprise a non-functional GULO protein and do not have the ability to synthesize ascorbate. However, as disclosed herein, human cells may be engineered to express a functional GULO protein to enable ascorbate synthesis ability. These functional GULO proteins may be expressed in human cells (or other cells that are unable to normally synthesize ascorbate) through conventional methods of cloning, such as genetically engineering cells to have genetic sequences that encode for a functional GULO protein.

The term “% w/w” or “% wt/wt” as used herein has its plain and ordinary meaning as understood in light of the specification and refers to a percentage expressed in terms of the weight of the ingredient or agent over the total weight of the composition multiplied by 100. The term “% v/v” or “% vol/vol” as used herein has its plain and ordinary meaning as understood in the light of the specification and refers to a percentage expressed in terms of the liquid volume of the compound, substance, ingredient, or agent over the total liquid volume of the composition multiplied by 100.

### Stem Cells

The term “totipotent stem cells” (also known as omnipotent stem cells) as used herein has its plain and ordinary meaning as understood in light of the specification and are stem cells that can differentiate into embryonic and extra-embryonic cell types. Such cells can construct a complete, viable organism. These cells are produced from the fusion of an egg and sperm cell. Cells produced by the first few divisions of the fertilized egg are also totipotent.

The term “embryonic stem cells (ESCs),” also commonly abbreviated as ES cells, as used herein has its plain and ordinary meaning as understood in light of the specification and refers to cells that are pluripotent and derived from the inner cell mass of the blastocyst, an early-stage embryo. For purpose of the present disclosure, the term “ESCs” is used broadly sometimes to encompass the embryonic germ cells as well.

The term “pluripotent stem cells (PSCs)” as used herein has its plain and ordinary meaning as understood in light of the specification and encompasses any cells that can differentiate into nearly all cell types of the body, i.e., cells derived from any of the three germ layers (germinal epithelium), including endoderm (interior stomach lining, gastrointestinal tract, the lungs), mesoderm (muscle, bone, blood, urogenital), and ectoderm (epidermal tissues and nervous system). PSCs can be the descendants of inner cell mass cells of the preimplantation blastocyst or obtained through induction of a non-pluripotent cell, such as an adult somatic cell, by forcing the expression of certain genes. Pluripotent stem cells can be derived from any suitable source. Examples of sources of pluripotent stem cells include mammalian sources, including human, rodent, porcine, and bovine.

The term “induced pluripotent stem cells (iPSCs),” also commonly abbreviated as iPS cells, as used herein has its plain and ordinary meaning as understood in light of the specification and refers to a type of pluripotent stem cells artificially derived from a normally non-pluripotent cell, such as an adult somatic cell, by inducing a “forced” expression of certain genes. hiPSC refers to human iPSCs. In some methods known in the art, iPSCs may be derived by transfection of certain stem cell-associated genes into non-pluripotent cells, such as adult fibroblasts. Transfection may be achieved through viral transduction using viruses such as retroviruses or lentiviruses. Transfected genes may include the master transcriptional regulators Oct-3/4 (POU5F1) and Sox2, although other genes may enhance the efficiency of induction. After 3-4 weeks, small numbers of transfected cells begin to become morphologically and biochemically similar to pluripotent stem cells, and are typically isolated through morphological selection, doubling time, or through a reporter gene and antibiotic selection. As used herein, iPSCs include first generation iPSCs, second generation iPSCs in mice, and human induced pluripotent stem cells. In some methods, a retroviral system is used to transform human fibroblasts into pluripotent stem cells using four pivotal genes: Oct3/4, Sox2, Klf4, and c-Myc. In other methods, a lentiviral system is used to transform somatic cells with OCT4, SOX2, NANOG, and LIN28. Genes whose expression are induced in iPSCs include but are not limited to Oct-3/4 (POU5F1); certain members of the Sox gene family (e.g., Sox1, Sox2, Sox3, and Sox15); certain members of the Klf family (e.g., Klf1, Klf2, Klf4, and Klf5), certain members of the Myc family (e.g., C-myc, L-myc, and N-myc), Nanog, LIN28, Tert, Fbx15, ERas, ECAT15-1, ECAT15-2, Tcl1, β-Catenin, ECATI, Esg1, Dnmt3L, ECAT8, Gdf3, Fth117, Sal14, Rex1, UTF1, Stella, Stat3, Grb2, Prdm 14, Nr5a1, Nr5a2, or E-cadherin, or any combination thereof.

The term “precursor cell” as used herein has its plain and ordinary meaning as understood in light of the specification and encompasses any cells that can be used in methods described herein, through which one or more precursor cells acquire the ability to renew itself or differentiate into one or more specialized cell types. In some embodiments, a precursor cell is pluripotent or has the capacity to becoming pluripotent. In some embodiments, the precursor cells are subjected to the treatment of external factors (e.g., growth factors) to acquire pluripotency. In some embodiments, a precursor cell can be a totipotent (or omnipotent) stem cell; a pluripotent stem cell (induced or non-induced); a multipotent stem cell; an oligopotent stem cells and a unipotent stem cell. In some embodiments, a precursor cell can be from an embryo, an infant, a child, or an adult. In some embodiments, a precursor cell can be a somatic cell subject to treatment such that pluripotency is conferred via genetic manipulation or protein/peptide treatment. Precursor cells include embryonic stem cells (ESC), embryonic carcinoma cells (ECs), and epiblast stem cells (EpiSC).

In some embodiments, one step is to obtain stem cells that are pluripotent or can be induced to become pluripotent. In some embodiments, pluripotent stem cells are derived from embryonic stem cells, which are in turn derived from totipotent cells of the early mammalian embryo and are capable of unlimited, undifferentiated proliferation in vitro. Embryonic stem cells are pluripotent stem cells derived from the inner cell mass of the blastocyst, an early-stage embryo. Methods for deriving embryonic stem cells from blastocytes are well known in the art. It would be understood by one of skill in the art that the methods and systems described herein are applicable to any stem cells.

Additional stem cells that can be used in embodiments in accordance with the present disclosure include but are not limited to those provided by or described in the database hosted by the National Stem Cell Bank (NSCB), Human Embryonic Stem Cell Research Center at the University of California, San Francisco (UCSF); WISC cell Bank at the Wi Cell Research Institute; the University of Wisconsin Stem Cell and Regenerative Medicine Center (UW-SCRMC); Novocell, Inc. (San Diego, Calif.); Cellartis AB (Goteborg, Sweden); ES Cell International Pte Ltd (Singapore); Technion at the Israel Institute of Technology (Haifa, Israel); and the Stem Cell Database hosted by Princeton University and the University of Pennsylvania. Exemplary embryonic stem cells that can be used in embodiments in accordance with the present disclosure include but are not limited to SA01 (SA001); SA02 (SA002); ES01 (HES-1); ES02 (HES-2); ES03 (HES-3); ES04 (HES-4); ES05 (HES-5); ES06 (HES-6); BG01 (BGN-01); BG02 (BGN-02); BG03 (BGN-03); TE03 (13); TE04 (14); TE06 (16); UCOI (HSF1); UC06 (HSF6); WA01 (HI); WA07 (H7); WA09 (H9); WA13 (H13); WA14 (H14). Exemplary human pluripotent cell lines include but are not limited to TkDA3-4, 1231A3, 317-D6, 317-A4, CDH1,5-T-3, 3-34-1, NAFLD27, NAFLD77, NAFLD150, WD90, WD91, WD92, L20012, C213, 1383D6, FF, or 317-12 cells.

In developmental biology, cellular differentiation is the process by which a less specialized cell becomes a more specialized cell type. As used herein, the term “directed differentiation” describes a process through which a less specialized cell becomes a particular specialized target cell type. The particularity of the specialized target cell type can be determined by any applicable methods that can be used to define or alter the destiny of the initial cell. Exemplary methods include but are not limited to genetic manipulation, chemical treatment, protein treatment, and nucleic acid treatment.

In some embodiments, an adenovirus can be used to transport the requisite four genes, resulting in iPSCs substantially identical to embryonic stem cells. Since the adenovirus does not combine any of its own genes with the targeted host, the danger of creating tumors is eliminated. In some embodiments, non-viral based technologies are employed to generate iPSCs. In some embodiments, reprogramming can be accomplished via plasmid without any virus transfection system at all, although at very low efficiencies. In other embodiments, direct delivery of proteins is used to generate iPSCs, thus eliminating the need for viruses or genetic modification. In some embodiment, generation of mouse iPSCs is possible using a similar methodology: a repeated treatment of the cells with certain proteins channeled into the cells via poly-arginine anchors was sufficient to induce pluripotency. In some embodiments, the expression of pluripotency induction genes can also be increased by treating somatic cells with FGF2 under low oxygen conditions.

The term “feeder cell” as used herein has its plain and ordinary meaning as understood in light of the specification and refers to cells that support the growth of pluripotent stem cells, such as by secreting growth factors into the medium or displaying on the cell surface. Feeder cells are generally adherent cells and may be growth arrested. For example, feeder cells are growth-arrested by irradiation (e.g. gamma rays), mitomycin-C treatment, electric pulses, or mild chemical fixation (e.g. with formaldehyde or glutaraldehyde). However, feeder cells do not necessarily have to be growth arrested. Feeder cells may serve purposes such as secreting growth factors, displaying growth factors on the cell surface, detoxifying the culture medium, or synthesizing extracellular matrix proteins. In some embodiments, the feeder cells are allogeneic or xenogeneic to the supported target stem cell, which may have implications in downstream applications. In some embodiments, the feeder cells are mouse cells. In some embodiments, the feeder cells are human cells. In some embodiments, the feeder cells are mouse fibroblasts, mouse embryonic fibroblasts, mouse STO cells, mouse 3T3 cells, mouse SNL 76/7 cells, human fibroblasts, human foreskin fibroblasts, human dermal fibroblasts, human adipose mesenchymal cells, human bone marrow mesenchymal cells, human amniotic mesenchymal cells, human amniotic epithelial cells, human umbilical cord mesenchymal cells, human fetal muscle cells, human fetal fibroblasts, or human adult fallopian tube epithelial cells. In some embodiments, conditioned medium prepared from feeder cells is used in lieu of feeder cell co-culture or in combination with feeder cell co-culture. In some embodiments, feeder cells are not used during the proliferation of the target stem cells.

### Differentiation of PSCs

Known methods for producing definitive endoderm from pluripotent cells (e.g., iPSCs or ESCs) are applicable to the methods described herein. In some embodiments, pluripotent cells are derived from a morula. In some embodiments, pluripotent stem cells are stem cells. Stem cells used in these methods can include, but are not limited to, embryonic stem cells or induced pluripotent stem cells. Embryonic stem cells can be derived from the embryonic inner cell mass or from the embryonic gonadal ridges. Embryonic stem cells or germ cells can originate from a variety of animal species including, but not limited to, various mammalian species including humans. In some embodiments, human embryonic stem cells are used to produce definitive endoderm. In some embodiments, human embryonic germ cells are used to produce definitive endoderm. In some embodiments, iPSCs are used to produce definitive endoderm. In some embodiments, human iPSCs (hiPSCs) are used to produce definitive endoderm.

In some embodiments, PSCs, such as ESCs and iPSCs, undergo directed differentiation into embryonic germ layer cells, organ tissue progenitor cells, and then into tissue such as liver tissue or any other biological tissue. In some embodiments, the directed differentiation is done in a stepwise manner to obtain each of the differentiated cell types where molecules (e.g. growth factors, ligands, agonists, antagonists) are added sequentially as differentiation progresses. In some embodiments, the directed differentiation is done in a non-stepwise manner where molecules (e.g. growth factors, ligands, agonists, antagonists) are added at the same time. In some embodiments, directed differentiation is achieved by selectively activating certain signaling pathways in the PSCs or any downstream cells.

In some embodiments, the embryonic stem cells or germ cells or iPSCs are treated with one or more small molecule compounds, activators, inhibitors, or growth factors for a time that is, is about, is at least, is at least about, is not more than, or is not more than about, 6 hours, 12 hours, 18 hours, 24 hours, 36 hours, 48 hours, 60 hours, 72 hours, 84 hours, 96 hours, 120 hours, 150 hours, 180 hours, 240 hours, 300 hours or any time within a range defined by any two of the aforementioned times, for example 6 hours to 300 hours, 24 hours to 120 hours, 48 hours to 96 hours, 6 hours to 72 hours, or 24 hours to 300 hours. In some embodiments, more than one small molecule compounds, activators, inhibitors, or growth factors are added. In these cases, the more than one small molecule compounds, activators, inhibitors, or growth factors can be added simultaneously or separately.

In some embodiments, the embryonic stem cells or germ cells or iPSCs are treated with one or more small molecule compounds, activators, inhibitors, or growth factors at a concentration that is, is about, is at least, is at least about, is not more than, or is not more than about, 10 ng/mL, 20 ng/mL, 50 ng/mL, 75 ng/mL, 100 ng/mL, 120 ng/mL, 150 ng/ml, 200 ng/mL, 500 ng/mL, 1000 ng/mL, 1200 ng/mL, 1500 ng/mL, 2000 ng/mL, 5000 ng/mL, 7000 ng/mL, 10000 ng/mL, or 15000 ng/mL, or any concentration that is within a range defined by any two of the aforementioned concentrations, for example, 10 ng/ml to 15000 ng/ml, 100 ng/ml to 5000 ng/mL, 500 ng/mL to 2000 ng/mL, 10 ng/mL to 2000 ng/mL, or 1000 ng/ml to 15000 ng/mL. In some embodiments, concentration of the one or more small molecule compounds, activators, inhibitors, or growth factors is maintained at a constant level throughout the treatment. In some embodiments, concentration of the one or more small molecule compounds, activators, inhibitors, or growth factors is varied during the course of the treatment. In some embodiments, more than one small molecule compounds, activators, inhibitors, or growth factors are added. In these cases, the more than one small molecule compounds, activators, inhibitors, or growth factors can differ in concentrations.

In some embodiments, the ESCs or iPSCs, or the ESCs, germ cells, or iPSCs are cultured in growth media that supports the growth of stem cells. In some embodiments, the ESCs or iPSCs, or the ESCs, germ cells, or iPSCs, are cultured in stem cell growth media. In some embodiments, the stem cell growth media is RPMI 1640, DMEM, DMEM/F12, or Advanced DMEM/F12. In some embodiments, the stem cell growth media comprises fetal bovine serum (FBS). In some embodiments, the stem cell growth media comprises FBS at a concentration that is, is about, is at least, is at least about, is not more than, or is not more than about, 0%, 0.1%, 0.2%, 0.3%, 0.4%, 0.5%, 0.6%, 0.7%, 0.8%, 0.9%, 1%, 2%, 3%, 4%, 5%, 6%, 7%, 8%, 9%, 10%, 11%, 12%, 13%, 14%, 15%, 16%, 17%, 18%, 19%, or 20%, or any percentage within a range defined by any two of the aforementioned concentrations, for example 0% to 20%, 0.2% to 10%, 2% to 5%, 0% to 5%, or 2% to 20%. In some embodiments, the stem cell growth media does not contain xenogeneic components. In some embodiments, the growth media comprises one or more small molecule compounds, activators, inhibitors, or growth factors.

In some embodiments, populations of cells enriched in definitive endoderm cells are used. In some embodiments, the definitive endoderm cells are isolated or substantially purified. In some embodiments, the isolated or substantially purified definitive endoderm cells express one or more (e.g. at least 1, 3) of SOX17, FOXA2, or CXRC4 markers to a greater extent than one or more (e.g. at least 1, 3, 5) of OCT4, AFP, TM, SPARC, or SOX7 markers.

In some embodiments, pluripotent stem cells are prepared from somatic cells. In some embodiments, pluripotent stem cells are prepared from biological tissue obtained from a biopsy. In some embodiments, the pluripotent stem cells are cryopreserved. In some embodiments, the somatic cells are cryopreserved. In some embodiments, pluripotent stem cells are prepared from PBMCs. In some embodiments, human PSCs are prepared from human PBMCs. In some embodiments, pluripotent stem cells are prepared from cryopreserved PBMCs. In some embodiments, PBMCs are grown on a feeder cell substrate. In some embodiments, PBMCs are grown on a mouse embryonic fibroblast (MEF) feeder cell substrate. In some embodiments, PBMCs are grown on an irradiated MEF feeder cell substrate.

In some embodiments, stem cells are treated with one or more growth factors to differentiate to definitive endoderm cells. Such growth factors can include growth factors from the TGF-beta superfamily. In some embodiments, the one or more growth factors comprise the Nodal/Activin and/or the BMP subgroups of the TGF-beta superfamily of growth factors. In some embodiments, the one or more growth factors are selected from the group consisting of Nodal, Activin A, Activin B, BMP4, Wnt3a or combinations of any of these growth factors. In some embodiments, the stem cells are contacted with Activin A. In some embodiments, the stem cells are contacted with Activin A and BMP4.

In some embodiments, activin-induced definitive endoderm (DE) can further undergo anterior endoderm pattering, foregut specification and morphogenesis, dependent on FGF, Wnt, or retinoic acid, or any combination thereof, or on FGF, Wnt, BMP, or retinoic acid, or any combination thereof, and a liver culture system that promotes liver growth, morphogenesis and cytodifferentiation. In some embodiments, human PSCs are efficiently directed to differentiate in vitro into liver epithelium and mesenchyme. It will be understood that molecules such as growth factors can be added to any stage of the development to promote a particular type of hepatic tissue formation.

It will be understood by one of skill in the art that altering the concentration, expression or function of one or more Wnt signaling proteins in combination with altering the concentration, expression, or function of one or more FGF proteins can give rise to directed differentiation in accordance with the present disclosure. In some embodiments, cellular constituents associated with the FGF, Wnt, or retinoic acid (RA) signaling pathways, or with the FGF, Wnt, BMP, or retinoic acid (RA) signaling pathways, for example, natural inhibitors, antagonists, activators, or agonists of the pathways can be used to result in inhibition or activation of the FGF, Wnt, or retinoic acid signaling pathways, or of the FGF, Wnt, BMP, or retinoic acid signaling pathways. In some embodiments, siRNA and/or shRNA targeting cellular constituents associated with the FGF, Wnt, or retinoic acid signaling pathways, or the the FGF, Wnt, BMP, or retinoic acid signaling pathways, are used to inhibit or activate these pathways.

In some embodiments, pluripotent stem cells, definitive endoderm, posterior foregut spheroids, or downstream liver cell types are contacted with a Wnt signaling pathway activator or Wnt signaling pathway inhibitor. In some embodiments, the Wnt signaling pathway activator comprises a Wnt protein. In some embodiments, the Wnt protein comprises a recombinant Wnt protein. In some embodiments, the Wnt signaling pathway activator comprises Wnt1, Wnt2, Wnt2b, Wnt3, Wnt3a, Wnt4, Wnt5a, Wnt5b, Wnt6, Wnt7a, Wnt7b, Wnt8a, Wnt8b, Wnt9a, Wnt9b, Wnt10a, Wnt10b, Wnt11, Wnt16, BML 284, IQ-1, WAY 262611, or any combination thereof. In some embodiments, the Wnt signaling pathway activator comprises a GSK3 signaling pathway inhibitor. In some embodiments, the Wnt signaling pathway activator comprises CHIR99021, CHIR 98014, AZD2858, BIO, AR-A014418, SB 216763, SB 415286, aloisine, indirubin, alsterpaullone, kenpaullone, lithium chloride, TDZD 8, or TWS119, or any combination thereof. In some embodiments, the Wnt signaling pathway inhibitor comprises C59, PNU 74654, KY-02111, PRI-724, FH-535, DIF-1, or XAV939, or any combination thereof. In some embodiments, the cells are not treated with a Wnt signaling pathway activator or Wnt signaling pathway inhibitor. The Wnt signaling pathway activator or Wnt signaling pathway inhibitor provided herein may be used in combination with any of the other growth factors, signaling pathway activators, or signaling pathway inhibitors provided herein.

In some embodiments, pluripotent stem cells, definitive endoderm, posterior foregut spheroids, or downstream liver cell types are contacted with an FGF signaling pathway activator. In some embodiments, the FGF signaling pathway activator comprises an FGF protein. In some embodiments, the FGF protein comprises a recombinant FGF protein. In some embodiments, the FGF signaling pathway activator comprises one or more of FGF1, FGF2, FGF3, FGF4, FGF4, FGF5, FGF6, FGF7, FGF8, FGF8, FGF9, FGF10, FGF11, FGF12, FGF13, FGF14, FGF15 (FGF19, FGF15/FGF19), FGF16, FGF17, FGF18, FGF20, FGF21, FGF22, or FGF23. In some embodiments, the cells are not treated with an FGF signaling pathway activator. The FGF signaling pathway activator provided herein may be used in combination with any of the other growth factors, signaling pathway activators, or signaling pathway inhibitors provided herein.

In some embodiments, pluripotent stem cells, definitive endoderm, posterior foregut spheroids, or downstream liver cell types are contacted with a retinoic acid signaling pathway activator or retinoic acid signaling pathway inhibitor. In some embodiments, the retinoic acid signaling pathway activator comprises retinoic acid, all-trans retinoic acid, 9-cis retinoic acid, CD437, EC23, BS 493, TTNPB, or AM580, or any combination thereof. In some embodiments, the retinoic acid signaling pathway inhibitor comprises guggulsterone. In some embodiments, the cells are not treated with a retinoic acid signaling pathway activator or retinoic acid signaling pathway inhibitor. The retinoic acid signaling pathway activator or retinoic acid signaling pathway inhibitor provided herein may be used in combination with any of the other growth factors, signaling pathway activators, or signaling pathway inhibitors provided herein.

In some embodiments, pluripotent stem cells are converted into liver cell types via a “one step” process. For example, one or more molecules that can differentiate pluripotent stem cells into DE culture (e.g., Activin A) are combined with additional molecules that can promote directed differentiation of DE culture (e.g., FGF4, CHIR99021, RA; or e.g., FGF4, Wnt, Noggin, RA) to directly treat pluripotent stem cells.

In some embodiments, iPSCs are expanded in cell culture. In some embodiments, pluripotent stem cells are expanded in a basement membrane matrix. In some embodiments, iPSCs are expanded in Matrigel. In some embodiments, the iPSCs are expanded in cell culture comprising a ROCK inhibitor (e.g. Y-27632). In some embodiments, the iPSCs are differentiated into definitive endoderm cells. In the iPSCs are differentiated into definitive endoderm cells by contacting the iPSCs with Activin A, BMP4, or both. In some embodiments, the iPSCs are contacted with a concentration of Activin A that is, is about, is at least, is at least about, is not more than, or is not more than about, 10, 20, 30, 40, 50, 60, 70, 80, 90, 100, 110, 120, 130, 140, 150, 160, 170, 180, 190, or 200 ng/ml, or any concentration of Activin A within a range defined by any two of the aforementioned concentrations, for example, 10 to 200 ng/ml, 10 to 100 ng/ml, 100 to 200 ng/mL, or 50 to 150 ng/mL. In some embodiments, the pluripotent stem cells are contacted with Activin A at a concentration of 100 ng/ml or about 100 ng/ml. In some embodiments, the iPSCs are contacted with a concentration of BMP4 that is, is about, is at least, is at least about, is not more than, or is not more than about, 1, 2, 3, 4, 5, 6, 7, 8, 9, 10, 20, 30, 40, 50, 60, 70, 80, 90, 100, 110, 120, 130, 140, 150, 160, 170, 180, 190, or 200 ng/ml, or any concentration of BMP4 within a range defined by any two of the aforementioned concentrations, for example, 1 to 200 ng/mL, 1 to 100 ng/ml, 25 to 200 ng/mL, 1 to 80 ng/ml, or 25 to 100 ng/mL. In some embodiments, the pluripotent stem cells are contacted with BMP4 at a concentration of 50 ng/ml or about 50 ng/mL.

In some embodiments, the PSCs are differentiated into definitive endoderm cells. In some embodiments, the PSCs are differentiated into posterior foregut cells. In some embodiments, the PSCs are differentiated into a liver organoid.

In some embodiments, any of the cells disclosed herein may be cryopreserved for later use. The cells can be cryopreserved according to methods generally known in the art.

### Gene Editing

In some embodiments, the iPSCs, definitive endoderm cells, posterior foregut spheroids, or organoids are genetically modified or edited according to methods known in the art. For example, gene editing using CRISPR nucleases such as Cas9 are explored in PCT Publications WO 2013/176772, WO 2014/093595, WO 2014/093622, WO 2014/093655, WO 2014/093712, WO 2014/093661, WO 2014/204728, WO 2014/204729, WO 2015/071474, WO 2016/115326, WO 2016/141224, WO 2017/023803, and WO 2017/070633, each of which is hereby expressly incorporated by reference in its entirety.

### Methods of Making Fetal-Like Liver Organoids

Methods of producing liver organoids have been explored previously in, for example, Ouchi et al. “Modeling Steatohepatitis in Humans with Pluripotent Stem Cell-Derived Organoids” Cell Metabolism (2019) 30 (2): 374-384; Shinozawa et al. “High-Fidelity Drug-Induced Liver Injury Screen Using Human Pluripotent Stem Cell Derived Organoids” Gastroenterology (2021) 160 (3): 831-846; PCT Publications WO 2018/085615, WO 2018/191673, WO 2018/226267, WO 2019/126626, WO 2020/023245, WO 2020/069285, WO 2020/243613, WO 2021/030373, and WO 2021/262676, each of which is hereby expressly incorporated by references in its entirety. Disclosure of liver organoid compositions and methods of making thereof are applicable to the human liver organoids (HLOs) described herein. These previously described methods generally result in “fetal-like” liver organoids, that exhibit characteristics of liver tissue early in development. For example, these fetal-like liver organoids express immature or fetal liver markers such as alpha fetoprotein (AFP), homeobox protein NANOG, and caudal type homeobox 2 (CDX2)

Embodiments of methods for producing fetal-like liver organoids are provided herein. In some embodiments, the methods comprise a) contacting definitive endoderm cells (DE) with an FGF signaling pathway activator and a Wnt signaling pathway activator for a first period of time; b) contacting the cells of step a) with the FGF signaling pathway activator, the Wnt signaling pathway activator, and a retinoic acid (RA) signaling pathway activator for a second period of time, thereby differentiating the DE to posterior foregut cells; and c) embedding the posterior foregut cells in a basement membrane matrix and culturing the posterior foregut spheroids for a third period of time to differentiate the posterior foregut cells to the fetal-like liver organoid. In some embodiments, the DE has been derived from pluripotent stem cells. In some embodiments, the pluripotent stem cells are embryonic stem cells and/or induced pluripotent stem cells. In some embodiments, the first period of time is, is about, is at least, is at least about, is not more than, or is not more than about, 0.5, 1, 2, 3, or 4 days, or a range defined by any two of the preceding values, for example 0.5-4, 1-4, 0.5-2, or 3-4 days. In some embodiments, the second period of time is, is about, is at least, is at least about, is not more than, or is not more than about 0.5, 1, or 2 days. In some embodiments, the third period of time is, is about, is at least, is at least about, is not more than, or is not more than about, 4, 5, 6, 7, 8, 9, 10, 11, 12, 13, 14, 15, 16, 17, 18, 19, 20, 21, 22, 23, 24, 25, 26, 27, 28, 29, or 30 days, or a range defined by any two of the preceding values, for example 4-30, 10-30, 20-30, 4-17, 4-12, or 10-25 days. In some embodiments, the basement membrane matrix is Matrigel. In some embodiments, the liver organoid, DE, and/or pluripotent stem cells are derived from a patient.

In some embodiments of the methods of making fetal-like liver organoids, the FGF signaling pathway activator is selected from the group consisting of FGF1, FGF2, FGF3, FGF4, FGF4, FGF5, FGF6, FGF7, FGF8, FGF8, FGF9, FGF10, FGF11, FGF12, FGF13, FGF14, FGF15, FGF16, FGF17, FGF18, FGF19, FGF20, FGF21, FGF22, and FGF23. In some embodiments, the FGF signaling pathway activator is FGF4. In some embodiments, the FGF signaling pathway activator is contacted at a concentration that is, is about, is at least, is at least about, is not more than, or is not more than about, 100, 150, 200, 250, 300, 350, 400, 450, 500, 550, 600, 650, 700, 750, 800, 850, 900, 950, or 1000 ng/mL, or any concentration within a range defined by any two of the aforementioned concentrations, including 100-1000 ng/ml, 100-500 ng/ml, 500-1000 ng/ml, 250-750 ng/mL, or 400-600 ng/mL. In some embodiments, the FGF signaling pathway activator is contacted at a concentration of 500 ng/ml or about 500 ng/ml.

In some embodiments of the methods of making fetal-like liver organoids, the Wnt signaling pathway activator is selected from the group consisting of Wnt1, Wnt2, Wnt2b, Wnt3, Wnt3a, Wnt4, Wnt5a, Wnt5b, Wnt6, Wnt7a, Wnt7b, Wnt8a, Wnt8b, Wnt9a, Wnt9b, Wnt10a, Wnt10b, Wnt11, Wnt16, BML 284, IQ-1, WAY 262611, CHIR99021, CHIR 98014, AZD2858, BIO, AR-A014418, SB 216763, SB 415286, aloisine, indirubin, alsterpaullone, kenpaullone, lithium chloride, TDZD 8, and TWS119. In some embodiments, the Wnt signaling pathway activator is CHIR99021. In some embodiments, the Wnt signaling pathway activator is contacted at a concentration that is, is about, is at least, is at least about, is not more than, or is not more than about, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, or 3.5 μM, or any concentration within a range defined by any two of the aforementioned concentrations, including 0.5-3.5 μM, 0.5-2 μM, 2-3.5 μM, 1-3 μM, or 1.5-2.5 μM. In some embodiments, the Wnt signaling pathway activator is contacted at a concentration of 2 μM or about 2 μM.

In some embodiments of the methods of making fetal-like liver organoids, the RA signaling pathway activator is selected from the group consisting of retinoic acid, all-trans retinoic acid, 9-cis retinoic acid, CD437, EC23, BS 493, TTNPB, and AM580. In some embodiments, the RA signaling pathway activator is RA. In some embodiments, the RA signaling pathway activator is contacted at a concentration that is, is about, is at least, is at least about, is not more than, or is not more than about, 1, 1.1, 1.2, 1.3, 1.4, 1.5, 1.6, 1.7, 1.8, 1.9, 2, 2.1, 2.2, 2.3, 2.4, 2.5, 2.6, 2.7, 2.9, or 3 μM, or any concentration within a range defined by any two of the aforementioned concentrations, including 1-3 μM, 1-2 μM, 2-3 μM, or 1.5-2.5 μM. In some embodiments, the RA signaling pathway activator is contacted at a concentration of 2 μM or about 2 μM.

### Maturation of Liver Organoids

Liver organoids prepared from pluripotent stem cells using previous methods resemble a fetal-like liver state. In some embodiments, this fetal-like liver organoid is characterized by expression of immature liver protein markers such as alpha fetoprotein (AFP) and caudal type homeobox 2 (CDX2).

Disclosed herein are methods of maturing fetal-like liver organoids by contacting the fetal-like liver organoids with a low/first concentration of bilirubin, thereby maturing the fetal-like organoids to mature liver organoids. In some embodiments, the low/first concentration of bilirubin is a human fetal physiological concentration of bilirubin. In some embodiments, the low/first concentration of bilirubin is, is about, is less than, or is less than about, 0.1 to 1 mg/L, 0.5 to 1 mg/L, or 1 mg/L. In some embodiments, the low/first concentration of bilirubin is, is about, is less than, or is less than about 0.1, 0.2, 0.3, 0.4, 0.5, 0.6, 0.7, 0.8, 0.9, or 1 mg/L, or any concentration within a range defined by any two of the aforementioned concentrations, for example, 0.1 to 1 mg/L, 0.1 to 0.5 mg/L, 0.5 to 1 mg/L, 0.3 to 0.7 mg/L, or 0.4 to 0.6 mg/L. In some embodiments, the low/first concentration of bilirubin is, is about, is less than, or is less than about, 0.1 to 3 mg/L, 0.5 to 3 mg/L, or 3 mg/L. In some embodiments, the low/first concentration of bilirubin is, is about, is less than, or is less than about 0.1, 0.2, 0.3, 0.4, 0.5, 0.6, 0.7, 0.8, 0.9, 1, 1.25, 1.5, 1.75, 2.0, 2.25, 2.5, 2.75 or 3.0 mg/L, or any concentration within a range defined by any two of the aforementioned concentrations, for example, 0.1 to 3 mg/L, 0.5 to 2.0 mg/L, 0.5 to 1.5 mg/L, 0.3 to 2.5 mg/L, or 0.5 to 1.75 mg/L. In some embodiments, the fetal-like liver organoid is differentiated from pluripotent stem cells (such as iPSCs or ESCs) according to a culture process that occurs over the span of 12, 13, 14, 15, 16, 17, 18, 19 or 20 days. In some embodiments, the fetal-like liver organoid is, is about, is at least, or is at least about, 12, 13, 14, 15, 16, 17, 18, 19, or 20 days old, or a range defined by any two of the aforementioned values, for example, 12-20, 14-16, or 15-18 days old, when used in the methods disclosed herein. In some embodiments, the fetal-like liver organoids are contacted with the low/first concentration of bilirubin for a period of time that is, is about, is at least, or is at least about, 12, 13, 14, 15, 16, 17, 18, 19, or 20 days, or a range defined by any two of the aforementioned values, for example, 12-20, 14-16, or 15-18 days, to mature into the mature liver organoids.

In some embodiments, the resultant mature liver organoid is smaller and/or less circular than a fetal-like liver organoid. In some embodiments, the mature liver organoid exhibits luminal projections that resemble bile canaliculi. In some embodiments, the mature liver organoid expresses reduced levels of AFP, CDX2, NANOG, or any combination thereof, relative to the fetal-like liver organoid. In some embodiments, the mature liver organoid expresses increased levels of ALB, SLC4A2, or HO-1, or any combination thereof, relative to the fetal-like liver organoid. In some embodiments, the mature liver organoid expresses CYP2E1, CYP7A1, PROX1, MRP3, MRP3, or OATP2, or any combination thereof. In some embodiments, the mature liver organoid exhibits increased CYP3A4 and CYP1A2 activity relative to the fetal-like liver organoid.

In some embodiments, the mature liver organoid may be engineered to express a functional GULO protein, which improves organoid viability and function as disclosed herein. In some embodiments, the mature liver organoid comprises a functional GULO protein and/or a gene or mRNA, or both, that encodes for the functional GULO protein, whereby the mature liver organoid is able to synthesize ascorbate. In some embodiments, the functional GULO protein is murine GULO (mGULO). However, the functional GULO may alternatively be derived from any other animal species that comprises a functional GULO protein. In some embodiments, the gene that encodes for the functional GULO protein is conditionally expressed. In some embodiments, the gene is conditionally expressed using a tetracycline inducible system or any other system for conditional expression generally known in the art. In some embodiments, the mature liver organoid is engineered with the gene that encodes for the functional GULO protein using CRISPR or any other method of genetic engineering generally known in the art. In some embodiments, the gene or mRNA, or both, that encodes for the functional GULO protein is introduced to the mature liver organoid by transfection. In some embodiments, the mature liver organoid comprising the functional GULO protein expresses increased levels of NRF2 relative to the fetal-like liver organoid or a mature liver organoid that does not comprise the functional GULO protein. In some embodiments, the mature liver organoid comprising the functional GULO protein expresses reduced levels of IL1B, IL6, or TNFa, or any combination thereof, relative to the fetal-like liver organoid or a mature liver organoid that does not comprise the functional GULO protein, optionally when cultured in ascorbate-depleted medium. In some embodiments, the mature liver organoid comprising the functional GULO protein exhibits reduced caspase-3 activity relative to the fetal-like liver organoid or a mature liver organoid that does not comprise the functional GULO protein, optionally when cultured in ascorbate-depleted medium. In some embodiments, the mature liver organoid comprising the functional GULO protein expresses increased levels of ALB relative to the fetal-like liver organoid or a mature liver organoid that does not comprise the functional GULO protein. In some embodiments, the mature liver organoid comprising the functional GULO protein resembles periportal liver tissue and expresses periportal liver markers. In some embodiments, the periportal markers comprise FAH, ALB, PAH, CPS1, HGD, or any combination thereof. In some embodiments, the mature liver organoid comprising the functional GULO protein exhibits increased CYP3A4 and CYP1A2 activity relative to the fetal-like liver organoid or a mature liver organoid that does not comprise the functional GULO protein. In some embodiments, the mature liver organoid comprising the functional GULO protein exhibits increased bilirubin conjugation activity relative to the fetal-like liver organoid or a mature liver organoid that does not comprise the functional GULO protein. In some embodiments, the mature liver organoid comprising the functional GULO protein exhibits increased viability in culture relative to the fetal-like liver organoid or a mature liver organoid that does not comprise the functional GULO protein.

In some embodiments, the fetal-like liver organoid is contacted with the low/first concentration of bilirubin in a hepatocyte culture medium. The compositions of these hepatocyte culture media (i.e. growth media that is designed for supporting hepatic tissues) is generally known in the art. In some embodiments, the hepatocyte culture medium comprises hepatocyte growth factor, oncostatin M, dexamethasone, or any combination thereof.

In some embodiments, the mature liver organoid is human. In some embodiments, the mature liver organoid comprises an inactive UGT1A1 gene, wherein the mature liver organoid is a model for Crigler-Najjar Syndrome. In some embodiments, the fetal-like liver organoid has been differentiated from pluripotent stem cells. In some embodiments, the pluripotent stem cells are embryonic stem cells or induced pluripotent stem cells. In some embodiments, the pluripotent stem cells comprise a functional GULO protein and/or a gene or mRNA, or both, that encodes for the functional GULO protein, whereby the pluripotent stem cells are able to synthesize ascorbate. Exemplary methods for producing liver organoids from pluripotent stem cells have been disclosed herein and are otherwise generally known in the art. In some embodiments, the fetal-like liver organoid has been made according to a method comprising: a) contacting definitive endoderm cells (DE) with an FGF signaling pathway activator and a Wnt signaling pathway activator for a first period of time; b) contacting the cells of step a) with the FGF signaling pathway activator, the Wnt signaling pathway activator, and a retinoic acid (RA) signaling pathway activator for a second period of time, thereby differentiating the DE to posterior foregut cells; and c) embedding the posterior foregut cells in a basement membrane matrix and culturing the posterior foregut spheroids for a third period of time to differentiate the posterior foregut cells to the fetal-like liver organoid.

Also disclosed herein are the fetal-like liver organoids provided herein. Also disclosed herein are the mature liver organoids provided herein.

### Hyperbilirubinemia Liver Organoids

As disclosed herein, a model liver organoid exhibiting characteristics of hyperbilirubinemia can be prepared. In some embodiments are methods of producing a hyperbilirubinemia liver organoid, the methods comprising contacting a liver organoid with a high/second concentration of bilirubin, thereby forming the hyperbilirubinemia liver organoid. In some embodiments, the liver organoid is a fetal-like liver organoid. In some embodiments, the liver organoid is a mature liver organoid (for example, any one of those disclosed herein prepared by contacting with a low/first concentration of bilirubin). In some embodiments, the liver organoid is, is about, is at least, or is at least about, 18, 19, 20, 21, 22, 23, 24, 25, 26, 27, 28, 29, 30, 31, 32, 33, 34, or 35 days old, or a range defined by any two of the aforementioned values, for example, 18-35, 18-30, 20-25, or 18-25 days when used in the methods disclosed herein. In some embodiments, the high/second concentration of bilirubin is, is about, is more than, or is more than about, 2-10 mg/L, 5-10 mg/L, 10 mg/L, or 20 mg/L. In some embodiments, the high/second concentration of bilirubin is, is about, is more than, or is more than about 2, 3, 4, 5, 6, 7, 8, 9, 10, 11, 12, 13, 14, 15, 16, 17, 18, 19, or 20 mg/L, or any concentration within a range defined by any two of the aforementioned concentrations, for example, 2 to 20 mg/L, 2 to 10 mg/L, 10 to 20 mg/L, 5 to 15 mg/L, or 8 to 12 mg/L. In some embodiments, the liver organoid is contacted with the high/second concentration of bilirubin for 1, 2, 3, 4, 5, 6, 7, 8, 9, or 10 days, or a range defined by any two of the aforementioned values, for example, 1-10, 1-5, 3-8, 5-10, or 7-10 days to form the hyperbilirubinemia liver organoid. In some embodiments, the hyperbilirubinemia liver organoid expresses elevated levels of UGT1A1 or NRF2, or both, relative to a liver organoid not treated with a high/second concentration of bilirubin. In some embodiments, the hyperbilirubinemia liver organoid is further contacted with a glucocorticoid antagonist to reduce the hyperbilirubinemia of the hyperbilirubinemia liver organoid. In some embodiments, contacting the hyperbilirubinemia liver organoid with the glucocorticoid antagonist increases expression of UGT1A1 and NRF2, and increases bilirubin conjugation activity in the hyperbilirubinemia liver organoid. In some embodiments, the glucocorticoid antagonist is ketoconazole, mifepristone, metyrapone, aminoglutethimide, or any combination thereof. In some embodiments, the hyperbilirubinemia liver organoid comprises a functional GULO protein and/or a gene or mRNA, or both, that encodes for the functional GULO protein, wherein the hyperbilirubinemia liver organoid is able to synthesize ascorbate. In some embodiments, the functional GULO protein is mGULO. However, the functional GULO may alternatively be derived from any other animal species that comprises a functional GULO protein. In some embodiments, the gene that encodes for the functional GULO protein is conditionally expressed. In some embodiments, the gene is conditionally expressed using a tetracycline inducible system or any other system for conditional expression generally known in the art. In some embodiments, the hyperbilirubinemia liver organoid comprises an inactive UGT1A1 gene, wherein the hyperbilirubinemia liver organoid is a model for Crigler-Najjar Syndrome. In some embodiments, the hyperbilirubinemia liver organoid is derived from embodiments of the fetal-like liver organoids disclosed herein. In some embodiments, the hyperbilirubinemia liver organoid is derived from embodiments of the liver organoids disclosed herein. In some embodiments, the hyperbilirubinemia liver organoid is produced from methods for producing liver organoids disclosed herein or generally known in the art.

Also disclosed herein are the hyperbilirubinemia liver organoids provided herein.

### Liver Organoids and Cells Expressing Exogenous GULO

Also disclosed herein are liver organoids comprising a functional GULO protein and/or a gene or mRNA, or both, that encodes for the functional GULO protein, whereby the liver organoid is able to synthesize ascorbate. In some embodiments, the functional GULO protein is mGULO. However, the functional GULO may alternatively be derived from any other animal species that comprises a functional GULO protein. In some embodiments, the gene that encodes for the functional GULO protein is conditionally expressed. In some embodiments, the gene is conditionally expressed using a tetracycline inducible system or any other system for conditional expression generally known in the art. In some embodiments, the liver organoid is engineered with the gene that encodes for the functional GULO protein using CRISPR or any other method of genetic engineering generally known in the art. In some embodiments, the gene or mRNA, or both, that encodes for the functional GULO protein is introduced to the mature liver organoid by transfection.

In some embodiments, the liver organoid comprising the functional GULO protein expresses increased levels of NRF2 relative to a liver organoid that does not comprise the functional GULO protein. In some embodiments, the liver organoid comprising the functional GULO protein expresses reduced levels of ILIB, IL6, or TNFa, or any combination thereof, relative to a liver organoid that does not comprise the functional GULO protein, optionally when cultured in ascorbate-depleted medium. In some embodiments, the liver organoid comprising the functional GULO protein exhibits reduced caspase-3 activity relative to a liver organoid that does not comprise the functional GULO protein, optionally when cultured in ascorbate-depleted medium. In some embodiments, the liver organoid comprising the functional GULO protein expresses increased levels of ALB relative to a liver organoid that does not comprise the functional GULO protein. In some embodiments, the liver organoid comprising the functional GULO protein resembles periportal liver tissue and expresses periportal liver markers. In some embodiments, the periportal markers comprise FAH, ALB, PAH, CPS1, HGD, or any combination thereof. In some embodiments, the liver organoid comprising the functional GULO protein exhibits increased CYP3A4 and CYP1A2 activity relative to a liver organoid that does not comprise the functional GULO protein. In some embodiments, the liver organoid comprising the functional GULO protein exhibits increased bilirubin conjugation activity relative to a liver organoid that does not comprise the functional GULO protein. In some embodiments, the liver organoid comprising the functional GULO protein exhibits increased viability in culture relative to a liver organoid that does not comprise the functional GULO protein.

In some embodiments, the liver organoid is human. In some embodiments, the liver organoid comprises an inactive UGT1A1 gene, wherein the liver organoid is a model for Crigler-Najjar Sydrome. In some embodiments, the liver organoid has been differentiated from pluripotent stem cells. In some embodiments, the pluripotent stem cells are embryonic stem cells or induced pluripotent stem cells. In some embodiments, the pluripotent stem cells have been engineered with the functional GULO protein and/or a gene or mRNA, or both, that encodes for the functional GULO protein, whereby the pluripotent stem cells are able to synthesize ascorbate.

Also disclosed herein are the liver organoids comprising a functional GULO protein and/or a gene or mRNA, or both, that encodes for the functional GULO protein as provided herein.

Also disclosed herein are liver cells comprising a functional GULO protein and/or a gene or mRNA, or both, that encodes for the functional GULO protein, whereby the liver cell is able to synthesize ascorbate. In some embodiments, the functional GULO protein is mGULO. However, the functional GULO may alternatively be derived from any other animal species that comprises a functional GULO protein. In some embodiments, the gene that encodes for the functional GULO protein is conditionally expressed. In some embodiments, the gene is conditionally expressed using a tetracycline inducible system or any other system for conditional expression generally known in the art. In some embodiments, the liver cell is engineered with the gene that encodes for the functional GULO protein using CRISPR or any other method of genetic engineering generally known in the art. In some embodiments, the gene or mRNA, or both, that encodes for the functional GULO protein is introduced to the liver cell by transfection.

Also disclosed herein are the liver cells comprising a functional GULO protein and/or a gene or mRNA, or both, that encodes for the functional GULO protein as provided herein.

Also disclosed herein are methods of increasing bilirubin conjugation and metabolism in a liver cell. In some embodiments, the methods comprise expressing a functional GULO protein and/or a gene or mRNA, or both, that encodes for the functional GULO protein, whereby the liver cell is able to synthesize ascorbate. In some embodiments, the liver cell may be part of a liver organoid, such as any of the liver organoids disclosed herein (including embodiments of the fetal-like liver organoids, mature liver organoids, and hyperbilirubinemia liver organoids provided herein).

### Methods of Use and Treatment

Disclosed herein are methods of treating hyperbilirubinemia in a subject in need thereof. In some embodiments, the methods comprise administering a glucocorticoid antagonist to the subject in need thereof. In some embodiments, the glucocorticoid antagonist is ketoconazole, mifepristone, metyrapone, aminoglutethimide, or any combination thereof.

Also disclosed herein are methods of screening for a compound or composition for the treatment of hyperbilirubinemia. In some embodiments, the methods comprise contacting a hyperbilirubinemia liver organoid with the compound or composition; and detecting an improvement in the hyperbilirubinemia of the hyperbilirubinemia liver organoid. In some embodiments, the hyperbilirubinemia liver organoid is any of the hyperbilirubinemia liver organoids disclosed herein. In some embodiments, the hyperbilirubinemia organoid comprises a functional GULO protein and/or a gene or mRNA, or both, that encodes for the functional GULO protein. In some embodiments, detecting the improvement comprises detecting an increase in expression of UGT1A1 or NRF2, or both, relative to an untreated hyperbilirubinemia liver organoid. In some embodiments, detecting an improvement comprises detecting a relative increase in conjugated bilirubin to unconjugated bilirubin relative to an untreated hyperbilirubinemia liver organoid.

Also disclosed herein are methods of treating a subject having a disease or disorder associated with bilirubin metabolism. In some embodiments, the methods comprise administering any of the liver organoids or liver cells disclosed herein. In some embodiments, the liver organoids or liver cells comprise a functional GULO protein and/or a gene or mRNA, or both, that encodes for the functional GULO protein. In some embodiments, the disease or disorder associated with bilirubin metabolism is hyperbilirubinemia, jaundice, Crigler-Najjar syndrome, Gilbert's syndrome, Dubin-Johnson syndrome, or Rotor syndrome.

Also disclosed herein are the liver organoids or liver cells disclosed herein for use in the manufacture of a medicament for the treatment of a disease or disorder associated with bilirubin metabolism. In some embodiments, the liver organoids or liver cells comprise a functional GULO protein and/or a gene or mRNA, or both, that encodes for the functional GULO protein. In some embodiments, the disease or disorder associated with bilirubin metabolism is hyperbilirubinemia, jaundice, Crigler-Najjar syndrome, Gilbert's syndrome, Dubin-Johnson syndrome, or Rotor syndrome.

Also disclosed herein are the liver organoids or liver cells disclosed herein for use in the treatment of a disease or disorder associated with bilirubin metabolism in a subject in need thereof. In some embodiments, the liver organoids or liver cells comprise a functional GULO protein and/or a gene or mRNA, or both, that encodes for the functional GULO protein. In some embodiments, the disease or disorder associated with bilirubin metabolism is hyperbilirubinemia, jaundice, Crigler-Najjar syndrome, Gilbert's syndrome, Dubin-Johnson syndrome, or Rotor syndrome.

### EXAMPLES

### Example 1. Material and Methods

### Animals

All animal experiments were conducted with the approval of an Institutional Review Board and Institutional Animal Care and Use Committee. Adult Gunn (Gunn-Ugtlalj/BluHsdRrrc) rats (breeding pairs, 9-12 weeks old) were obtained from the Rat Resource & Research Center (RRRC, Columbia, MO). Rats were housed in standard rat cages with woodchip bedding, maintained at a temperature of 20-24° C. and relative humidity of 45-55%, under a 12 h: 12 h light: dark cycle. All animals had ad libitum access to standard chow (Cincinnati Lab Supply, Cincinnati, OH) before study. All animals were treated in accordance with the guidelines and regulations of the institution.

### Maintenance of PSCs

A human iPSC line, 72.3 (RRID: CVCL_A1BW) was obtained from Cincinnati Children's Hospital Medical Center (CCHMC) Pluripotent Stem Cell Facility co-directed by CN. Mayhew and J M. Wells. Undifferentiated hiPSCs were cultured on Laminin-511 E8 fragment (Nippi) coated dishes in StemFit medium (Ajinomoto Company) with 100 ng/mL basic fibroblast growth factor (FGF; R&D Systems) at 37° C. in 5% CO2 with 95% air.

### Human Liver Organoid (HLO) Generation

Pluripotent stem cells were plated on a 24 well plate coated with Laminin iMatrix-511 Silk at a density of 2×10⁵ cells/well and maintained with StemFit media with Y-27632. On Day 2, the media was replaced with fresh StemFit media. The following day, the cells were treated with RPMI media mixed with Activin A and BMP4 to generate definitive endoderm. On Day 4, the media was replaced with RPMI, Activin A and 0.2% dFBS, which was changed to 2% dFBS on Day 5. From Day 6-8, the cells were fed with FGF4 and CHIR99021 in Advanced DMEM (supplemented with B27, N2, 10 mM HEPES, 2 mM L-glutamine, and gentamicin-amphotericin) to induce posterior foregut. On Day 9, the cells were dissociated into a single cell suspension using Accutase treatment. This single cell suspension was then mixed with 50% Matrigel and 50% EP media (Advanced DMEM/F12, B27/N2/HEPES/Glutamax, 5 ng/mL FGF2, 10 ng/mL VEGF, 3 μM CHIR99021, 500 nM A83-01, and 50 μg/mL ascorbic acid) and plated as 50 μl drops in a 6-well plate. These cells were fed with EP media every 48 hours for 4 days to generate organoids. These organoids were then treated with Advanced DMEM and retinoic acid (RA) every 48 hours for 4 days to specify the hepatic lineage. The organoids were then fed with hepatocyte culture medium (HCM), hepatocyte growth factor (HGF), Oncostatin M, and Dexamethasone every 3-4 days to generate HLOs.

Maturation of HLOs with Low Dose Bilirubin

For maturation induction in HLOs, the day 15 organoids were fed with complete HCM with supplements in addition to low dose bilirubin (1 mg/L). This treatment was continued until day 30 when the organoids were harvested. The brightfield images were captured on the KEYENCE BZ-X710 Fluorescence Microscope (Keyence), and the images were analyzed using ImageJ suite. RNA was isolated using the RNeasy mini kit (Qiagen, Hilden, Germany). Reverse transcription was carried out using the High-Capacity cDNA Reverse Transcription Kit (Thermo Fisher Scientific Inc.) according to manufacturer's protocol. qPCR was carried out using TaqMan gene expression master mix (Applied Biosystems) on a QuantStudio 5 Real-Time PCR System (Thermo Fisher Scientific Inc.). All primers and probe information for each target gene was obtained from the Universal ProbeLibrary Assay Design Center website (available on the World Wide Web at lifescience.roche.com/en_us/brands/universal-probe-library.html). For whole mount immunostains, the organoids were fixed in 4% PFA, permeabilized with 0.1% PBST, blocked with 5% Normal Donkey Serum in 0.1% PBST, and stained with the appropriate primary and secondary antibodies. The images were captured on the Nikon A1 inverted confocal microscope.

### Bilirubin and Drug Treatment

On day 27, the mature organoids were treated with bilirubin (1-10 mg/L), doxycycline (100 ng/mL; added 3 days prior to activate gene expression) and/or additionally with drugs such as hydrocortisone, dexamethasone, ketoconazole and mifepristone (1-2 μM for each) for 5 days and then harvested for downstream assays. Bilirubin assay was performed using a colorimetric kit (ab235627 from Abcam). RNA was isolated using the RNeasy mini kit (Qiagen, Hilden, Germany). Reverse transcription was carried out using the High-Capacity cDNA Reverse Transcription Kit (Thermo Fisher Scientific Inc.) according to manufacturer's protocol. qPCR was carried out using TaqMan gene expression master mix (Applied Biosystems) on a QuantStudio 5 Real-Time PCR System (Thermo Fisher Scientific Inc.). All primers and probe information for each target gene was obtained from the Universal ProbeLibrary Assay Design Center (available on the World Wide Web at lifescience.roche.com/en_us/brands/universal-probe-library.html). Images were captured on the KEYENCE BZ-X710 Fluorescence Microscope (Keyence).

Organoid Transplantation into the Portal Vein

HLOs were harvested on Day 27 and dissociated into organoid fragments by repeated pipetting, washed with PBS and resuspended with HCM containing 2% FBS and CEPT cocktail (50 nM chroman 1, 5 μM emricasan, 1:1000 polyamine, and 7 μM trans-ISRIB) to increase viability. The recipient rats were treated with a single dose of retrorsine (5 mg/kg) and tacrolimus (0.8 mg/kg) 4 days prior to the transplantation. A midline incision was drawn, the intestines pushed to one side, and a 32 g 1 inch needle was used to inject 3×10³ organoids (roughly 5×10⁵ cells) in a 200 μL infusion into the portal vein by clamping it down posteriorly to control bleeding. Excessive blood loss was prevented by application of a SURGICEL SNOW Absorbable Hemostat (Ethicon). The animal was then closed with 5-0 vicryl coated surgical sutures (Ethicon) and GLUture (Zoetis Inc.), and buprenorphine (0.1 mg/kg) was administered as an analgesic. The animal was then maintained on doxycycline (2 mg/kg) and tacrolimus injections every 3-4 days until the day of harvest. Blood was collected regularly by the retro-orbital method as needed.

### Live Cell Imaging and Functional Assay

For live imaging of organoids, the Celldiscoverer 7 (Zeiss) was used to image every 30 minutes for 7 days. Visualization of bilirubin conjugation was achieved with 5 μM fluorescent UnaG, which was incubated with HLO media and imaged for 2 days.

### RNA Sequencing and Analysis

RNA was isolated using the RNeasy mini kit (Qiagen). Reverse transcription was carried out using the High-Capacity cDNA Reverse Transcription Kit for RT-PCR (Applied Biosystems) according to manufacturer protocol. qPCR was carried out using TaqMan gene expression master mix (Applied Biosystems) on a QuantStudio 5 Real-Time PCR System (Applied Biosystems). All the samples were amplified with TaqMan Gene Expression Assays and normalized with 18S rRNA Endogenous Control. For RNA sequencing, the extracted RNA quality was evaluated with an Agilent 2100 Bioanalyzer (Agilent). A sequence library was prepared using a TruSeq Stranded mRNA kit (Illumina) and sequenced using NovaSeq 6000 (Illumina). Reads were aligned to human genome assembly hg38 and quantified using the quasi-mapper Salmon (v1.8.0). Gene-expression analysis was performed using the R Bioconductor package DESeq2 (v1.36.0). The read count matrix was normalized by size factors, and a variance stabilizing transformation (VST) was applied to the normalized expression data. The data was visualized using clusterProfiler (v.4.4.1) and pheatmap (v1.0.12) packages.

Alternatively, whole-transcriptome RNA sequencing of HLOs (including those treated with bilirubin and those treated with mifepristone) was performed using the services of Novogene Co., Ltd. (Beijing, China) on the Illumina NovaSeq platform from isolated total RNA. RNA sequencing parameters were 150 bp paired-end sequencing at a depth of 20M reads per samples. Fastq read files for each sample were obtained and then aligned using Salmon, a quasi-mapping tool that aligns and quantifies transcripts using RNA-seq data. Raw transcript counts and normalized transcripts per million (TPM) values were obtained and analyzed for differential expression with DESeq2. For differential expression, statistical and biological significance was set at P<0.05, FDR<0.05, log fold-change>1, with a minimum of 3 transcript counts in 3 of the 6 samples. For heatmap visualization and hierarchical clustering analysis, hclust and pheatmap were used respectively. Finally, the pathway analysis was carried out using biomaRt and org. Hs.eg.db in R version 4.0.3.

mGULO Editing

The murine GULO (L-gulonolactone oxidase) (mGULO) cDNA sequence was retrieved from NCBI. The 5′ linker and Kozak sequence were added to the start of the sequence and HA tags were added to the end of the sequence. Additionally, P2A-mCherry was added after the HA tag and a 3′ linker to the very end. The custom gene was then synthesized and cloned into the pAAVS1-NDi-CRISPRi (Gen1) PCSF #117 vector using the restriction sites AflII and Agel. The vector has a TetON system and a Neo′ selectable marker was then inserted using the Gateway technology.

mGULO iPSC Generation and Maintenance

The PCSF #117 vector with the modified GULO sequence was then inserted into the AAVS1 locus of a 72.3 iPSC cell line using a lentiviral mediated CRISPR/Cas9. The correct clones were then selected using G418. The surviving clones were then verified for correct insertion, random insertion and copy number using PCR, and verified by DNA sequencing. The edited iPSC was then plated on Laminin iMatrix-511 Silk coated cell culture plates and maintained with StemFit Basic04 Complete Type media with Y-27632. The cells were passaged every 4-7 days with Accutase until passage 40 (p40). mGULO HLOs were generated according to the HLO generation protocol as described herein. The mGULO protein expression was verified using a GLUO ELISA kit (MBS2890737 from MyBioSource, San Diego, CA).

mGULO HLO Treatment with Bilirubin and Bilirubin Visualization

On day 24, the mGLUO HLOs were treated with doxycycline (Dox) (100 ng/ml) to induce mGULO expression. Subsequently, on day 27, the mature organoids were treated with bilirubin and Dox for 5 days and then harvested for downstream assays. A bilirubin assay was performed to measure and visualize unconjugated and conjugated bilirubin using a colorimetric kit (ab235627) and UnaG, a green-to-dark photoswitching fluorescent protein that only fluoresces upon binding of bilirubin. Images were captured on the KEYENCE BZ-X710 Fluorescence Microscope (Keyence).

### ChIP-PCR and ChIP-qPCR

ChIP experiments were performed using the High Sensitivity ChIP Kit (Abcam). Briefly, organoids were fixed with PFA, and then whole chromatin was prepared and sonicated to an optimal size of 300 bp, which was confirmed by gel electrophoresis. Chromatin was used for immunoprecipitation with either EP300 antibody or IgGI isotype control. DNA fragments were amplified using custom primers for PCR and qPCR, and fold enrichment data were normalized to the immunoprecipitation from IgG controls.

### Protein Expression Assays

Albumin secretion was measured by collecting 200 μL of the supernatant from HLOs cultured in HCM and stored at −80° C. until use. The supernatant was assayed with Human Albumin ELISA Quantitation Set (Bethyl Laboratories) according to manufacturer instructions.

For murine GULO expression assay, the organoids were dissociated and washed with PBS. The cells were then lysed with RIPA Lysis and Extraction Buffer and Halt Protease and Phosphatase Inhibitor Cocktail (Thermo Scientific) to extract total protein and assayed with Mouse GULO/L-gulonolactone oxidase ELISA kit (MyBioSource.com) according to manufacturer instructions.

### Metabolite Assays

Bilirubin levels were measured by collecting the supernatant from HLOs treated with bilirubin and serum from rats. The supernatant and serum were assayed with Bilirubin Assay Kit (Total and Direct, Colorimetric) (abcam) and Bilirubin Assay Kit (Sigma-Aldrich) according to manufacturer instructions.

Cellular antioxidant levels were measured by harvesting the HLOs, washing in PBS, and plating them into a 96 well assay plate. The levels were then quantitated using Cellular Antioxidant Assay Kit (abcam) according to manufacturer instructions.

### Activity Assays

CYP3A4 and CYP1A2 assays were performed by harvesting HLOs, washing in PBS, plating them into a 96 well assay plate, and treating them with rifampicin and omeprazole, respectively, for 24 hours. The assays were then performed using P450-Glo CYP3A4 and CYP1A2 Assay (Promega) and normalized using CellTiter-Glo Luminescent Cell Viability Assay according to manufacturer instructions.

The apoptosis assay was carried out by lysing HLOs and assaying the lysate with a Caspase-3 Assay Kit (Colorimetric) (abcam) according to manufacturer instructions.

Rat serum was assayed with the Aspartate Aminotransferase (AST) Activity Assay Kit and Alanine Transaminase (ALT) Activity Assay Kit (Sigma-Aldrich).

### Quantification and Statistical Analysis

Statistical analyses were mainly performed using R software v4.2.0 with unpaired two-tailed Student's t-test, Dunn-Holland-Wolfe test, or Welch's test. Statistical analyses for stiffness measurements were performed using non-parametric Kruskal-Wallis and post hoc Dunn-Holland-Wolfe test. For comparisons between 2 unpaired groups, when groups were independent and the variances were unequal, non-parametric Brunner-Munzel test was performed, unless noted otherwise. P values<0.05 were considered statistically significant. N-value refers to biologically independent replicates. The image analyses were non-blinded.

### Example 2. Low Doses of Promote Maturation of Fetal-Like Liver Organoids

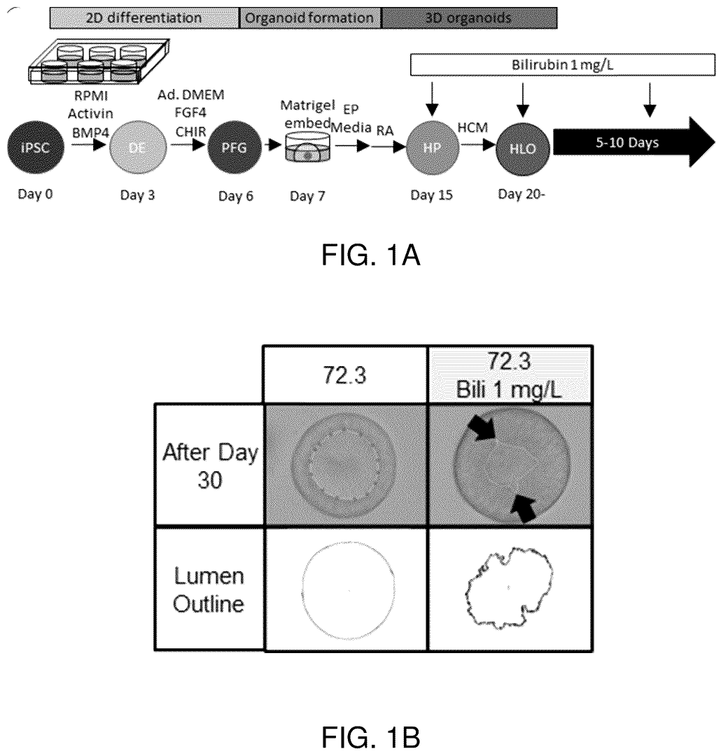

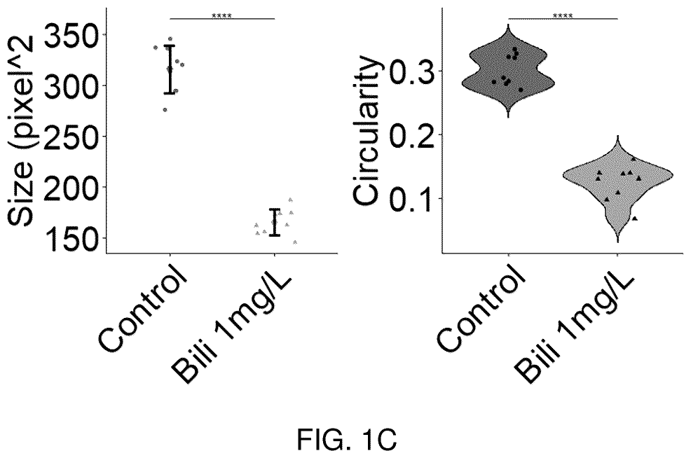

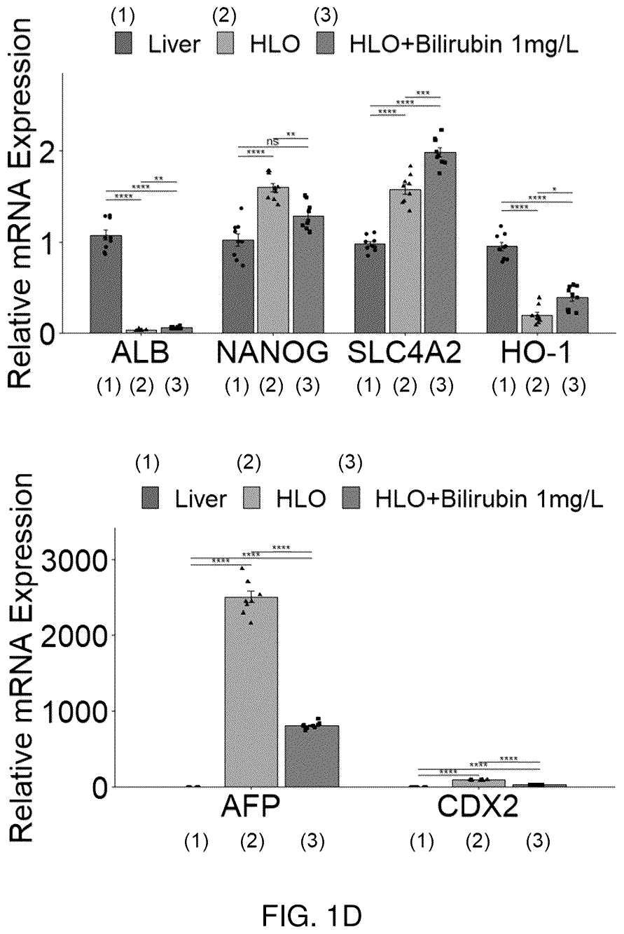

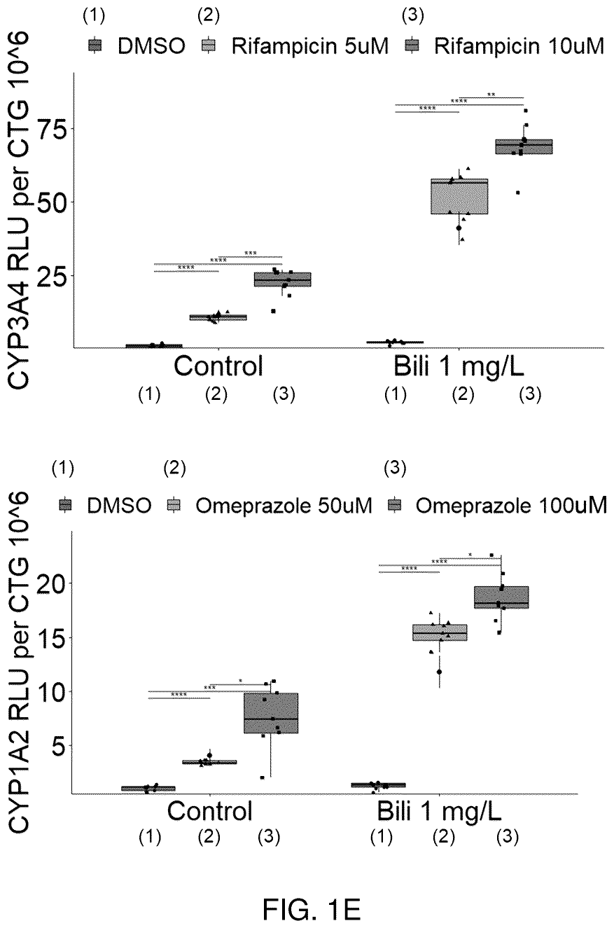

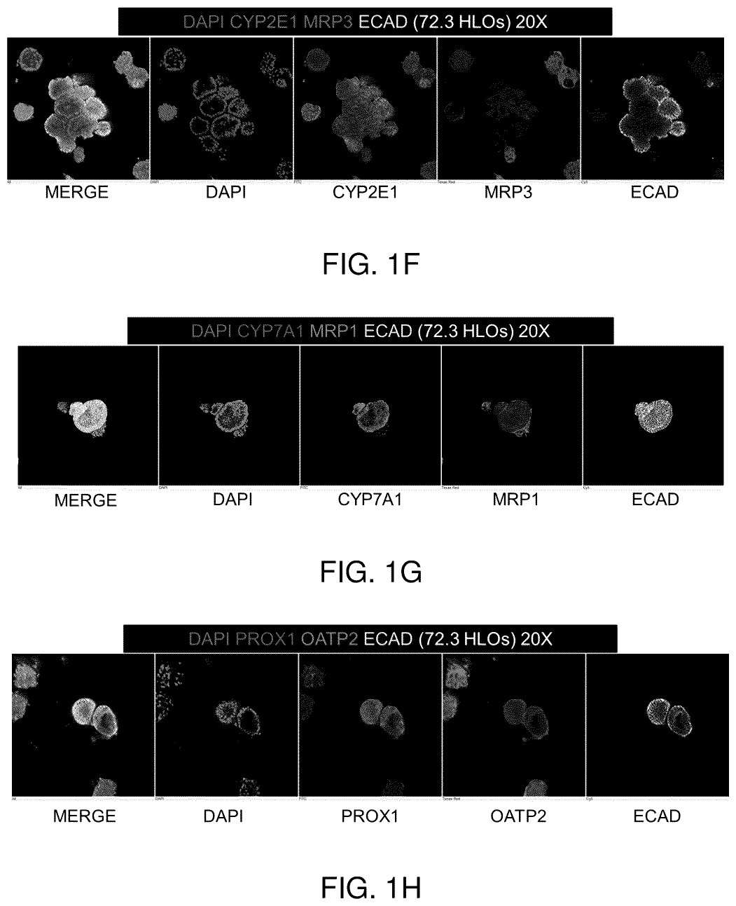

The role of bilirubin for liver development was investigated using human liver organoid models. FIG. 1A depicts an exemplary schematic for preparing liver organoids treated with a low concentration of bilirubin (e.g., 1 mg/L) resembling human fetal physiological concentrations (which is approximately 10 times less than the physiological concentrations in adults). The bilirubin is added to early organoids differentiated to a hepatic lineage. Exemplary methods of producing liver organoids have been explored previously in, for example, PCT Publications WO 2018/085615, WO 2018/191673, WO 2018/226267, WO 2019/126626, WO 2020/023245, WO 2020/069285, WO 2020/243613, WO 2021/030373, and WO 2021/262676, each of which is hereby expressly incorporated by references in its entirety. Based on these exemplary methods, the low concentration of bilirubin was added to liver organoids that form after retinoic acid induction of posterior foregut endoderm. The liver organoids may be cultured with bilirubin in a standard hepatocyte culture medium (HCM). For example, the hepatocyte culture medium may be supplemented with hepatocyte growth factors including but not limited to hepatocyte growth factor (HGF), oncostatin M, and/or dexamethasone. The liver organoids were contacted with growth medium containing 1 mg/L bilirubin for at least 5-10 days to promote liver organoid maturation.

FIG. 1B shows that the liver organoids matured with 1 mg/L bilirubin exhibited luminal projections resembling bile canaliculi, which are natural structures found in liver tissue. The resultant mature liver organoids exhibit lumens with smaller sizes and reduced circularity compared to the lumens of control liver organoids that were not treated with bilirubin (FIG. 1C).

Gene expression quantification by RT-qPCR of bilirubin-treated liver organoids revealed that these organoids exhibited increased expression of mature liver markers such as albumin (ALB), solute carrier family 4 member 2 (SLC4A2), and heme oxygenase-1 (HO-1), and reduced expression of immature or fetal liver markers such as alpha fetoprotein (AFP), homeobox protein NANOG, and caudal type homeobox 2 (CDX2) relative to untreated organoids (FIG. 1D).

The drug metabolic capacity of the bilirubin-treated organoids was assessed by measuring cytochrome P450 3A4 (CYP3A4) and cytochrome P450 1A2 (CYP1A2) activity after treatment with rifampicin and omeprazole. The bilirubin-treated organoids exhibited increased cytochrome activity relative to control untreated organoids (FIG. 1E).

Immunofluorescence microscopy showed that the bilirubin-treated organoids expressed mature liver enzymes and transport proteins, including cytochrome P450 2E1 (CYP2E1), cytochrome P450 7A1 (CYP7A1), multidrug resistance protein 1 (MPR1), multidrug resistance associated protein 3 (MRP3), prospero homeobox 1 (PROX1), and organic anion transporting polypeptide 2 (OATP2) (FIGS. 1F-H).

Therefore, the use of a low concentration of bilirubin resembling physiological conditions induces maturation of liver organoids in culture.

### Example 3. Ascorbate Promotes Liver Organoid Viability and GULO Induces a Periportal-Like Identity

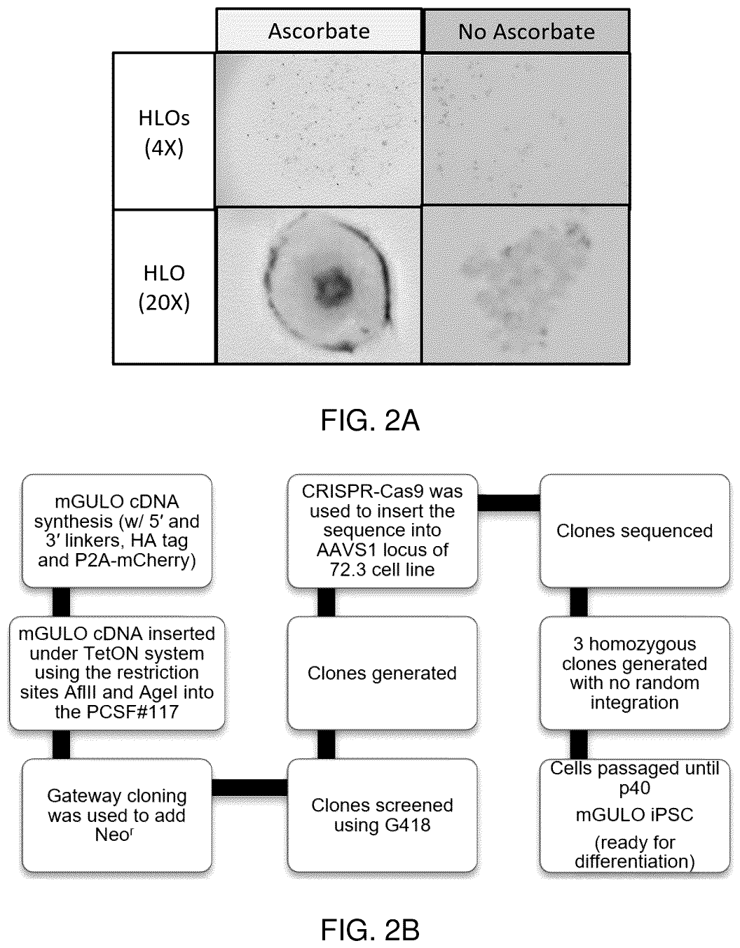

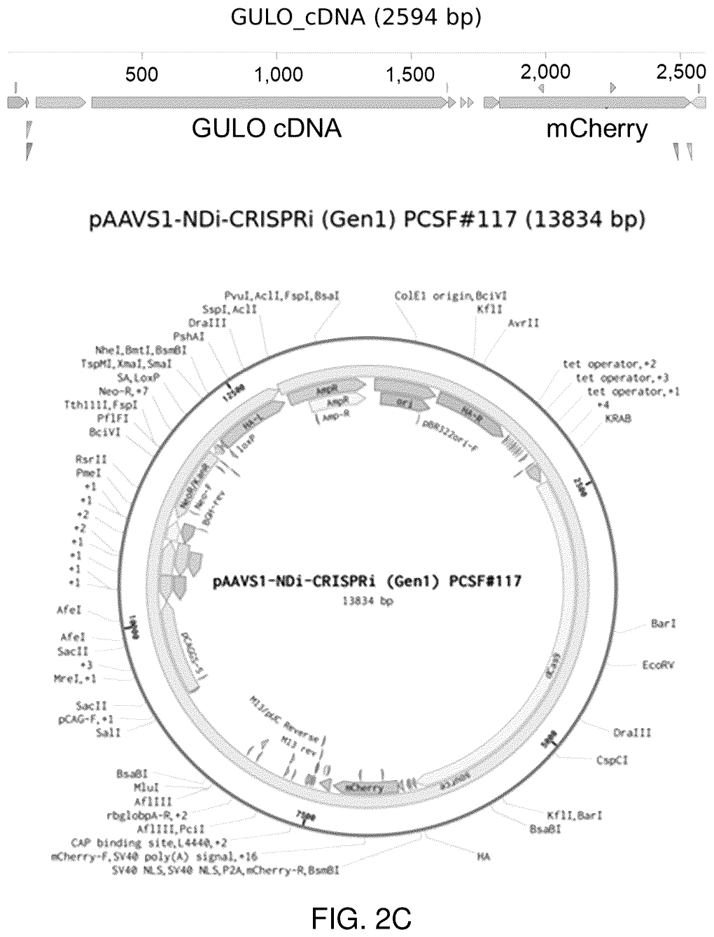

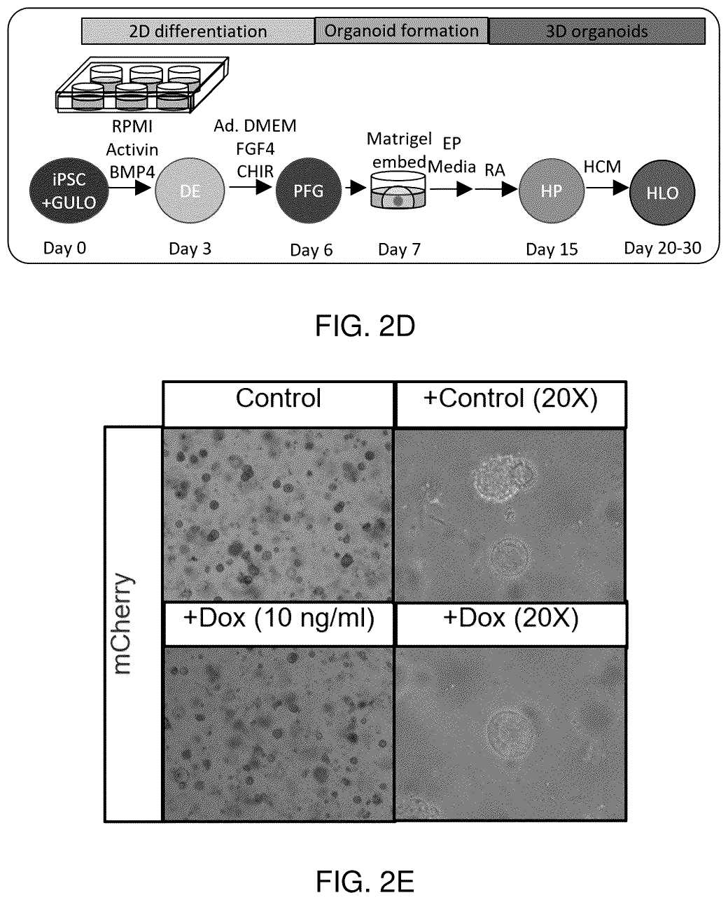

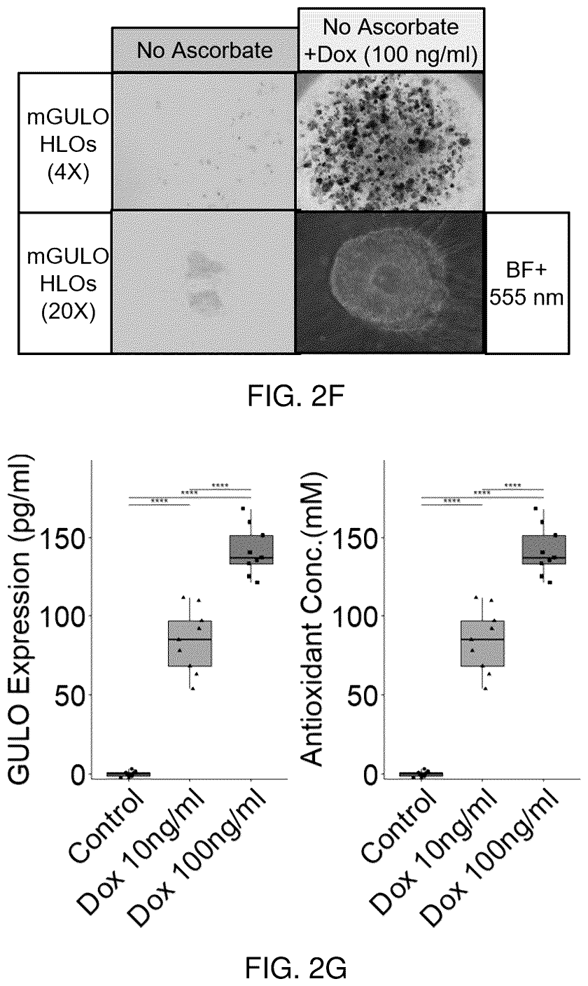

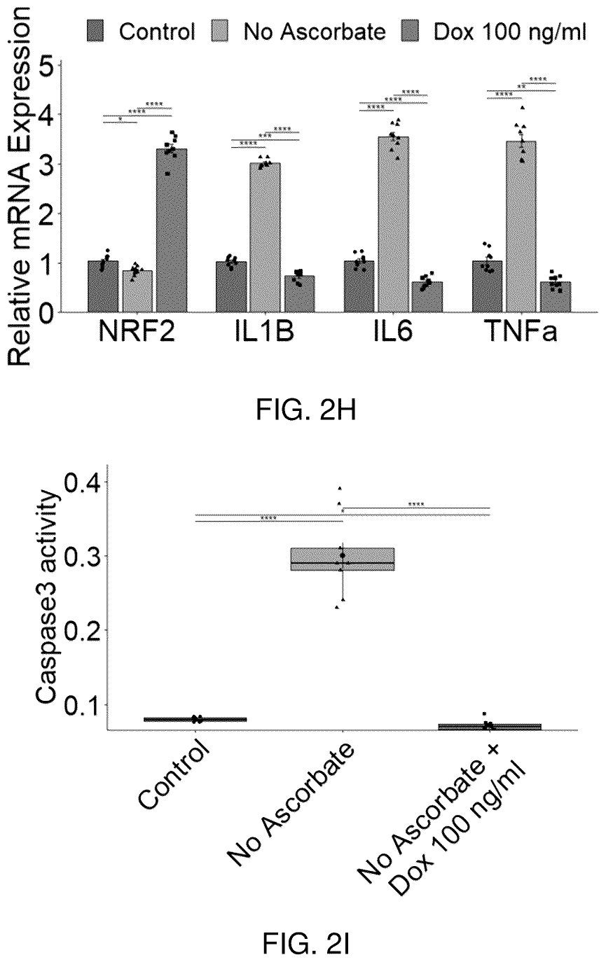

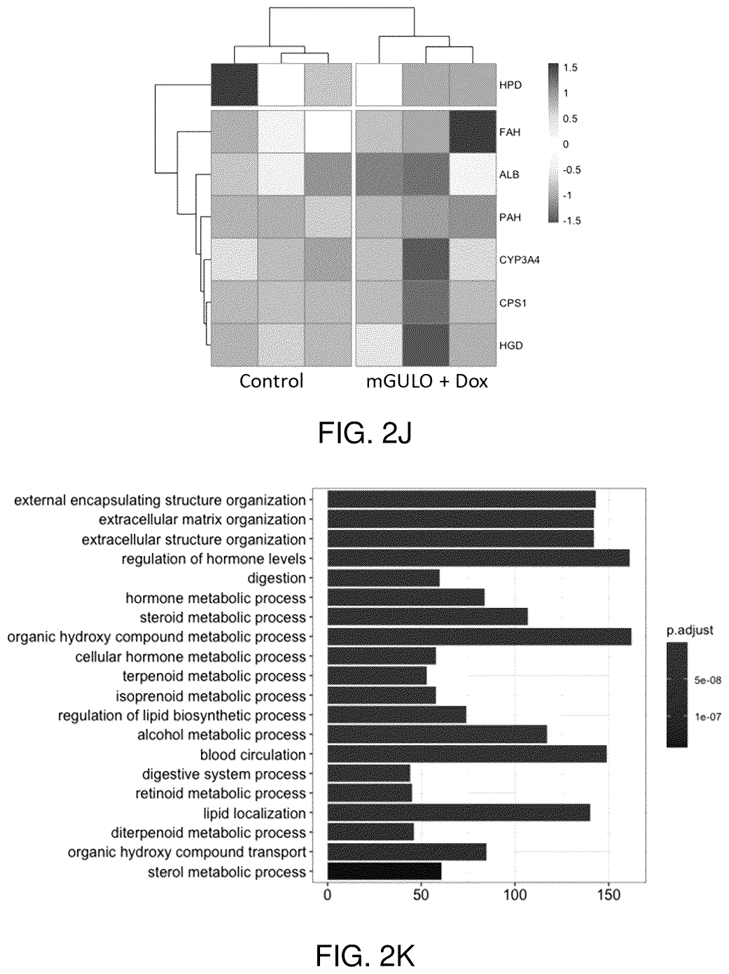

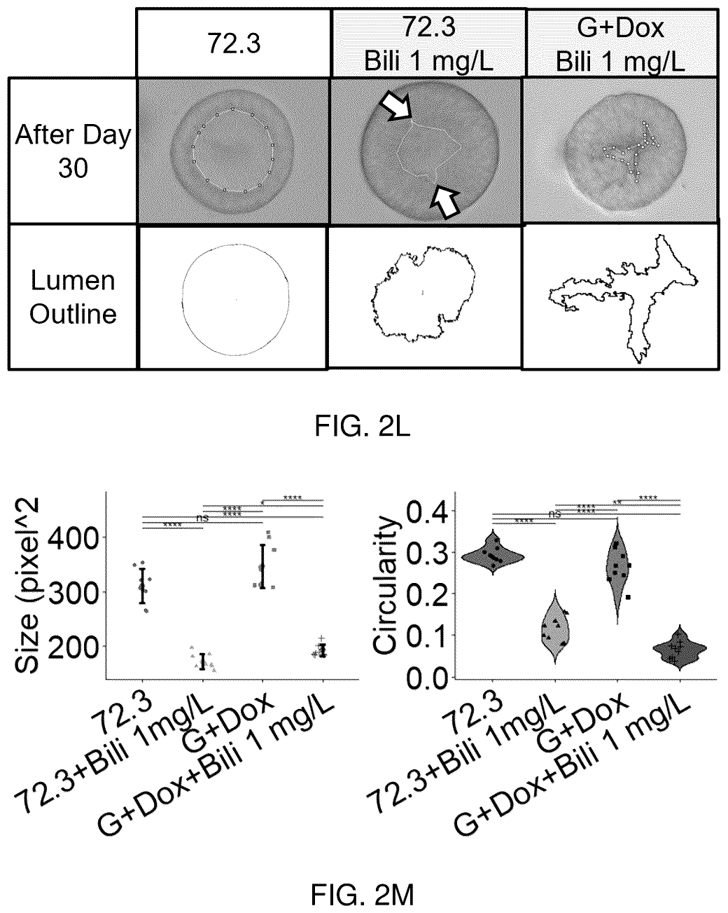

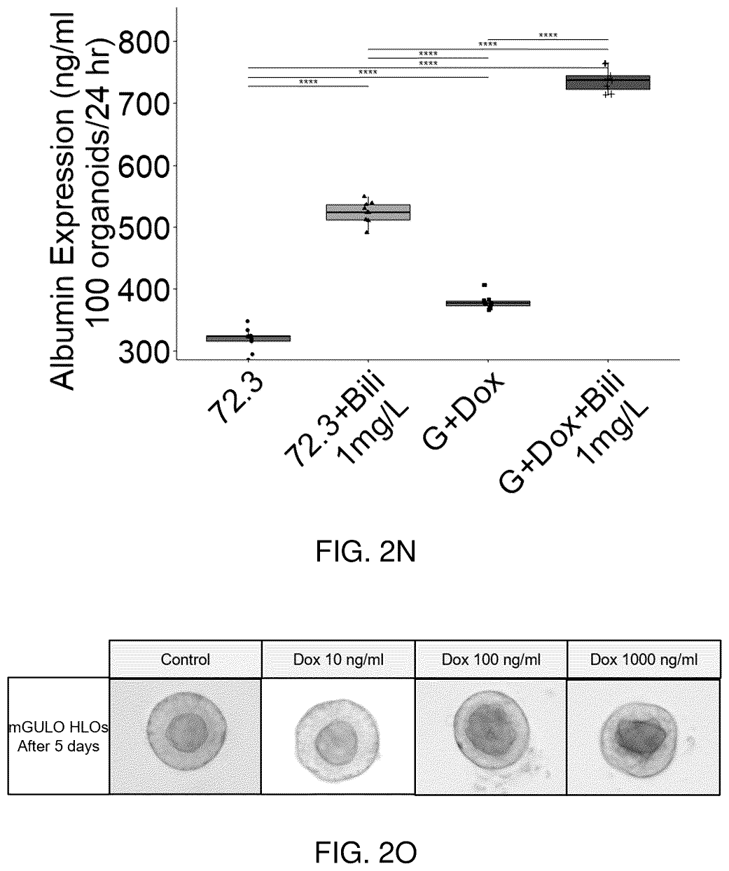

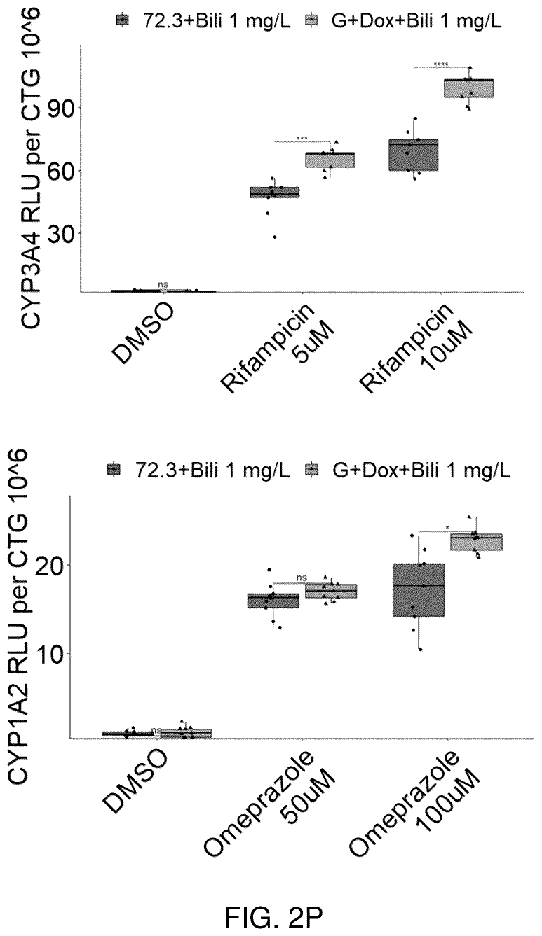

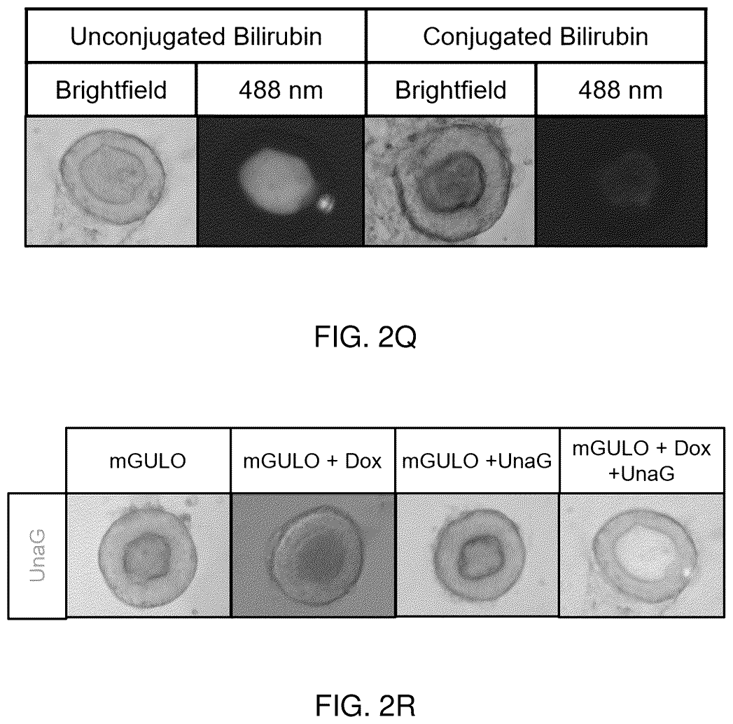

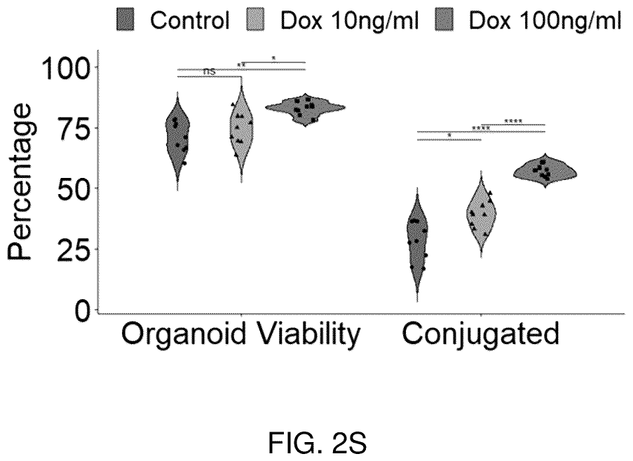

The role of ascorbate (vitamin C) for liver development was investigated using human liver organoid models. Liver organoids cultured in media lacking ascorbate exhibited loss of viability and apoptosis (FIG. 2A). This result was expected, as ascorbate is an essential nutrient that cannot be synthesized by human cells naturally due to a non-functional L-gulonolactone oxidase (GULO) enzyme.

Unlike humans and some other mammals such as Guinea pigs, many other mammals have a functional GULO gene and are able to enzymatically synthesize ascorbate. As ascorbate is important for liver development, it was hypothesized that expressing exogenous GULO in liver organoids would improve their growth and maturation in culture.

FIG. 2B depicts an exemplary schematic for genetically engineering human pluripotent stem cells with a GULO expression construct driven by a TetOn conditional expression system using CRISPR/Cas9. It is envisioned that alternative methods of exogenously introducing GULO to cells may be used. Furthermore, although the GULO gene from mouse (mGULO) was used herein, analogous functional GULO genes from other mammals may also be used. FIG. 2C shows an embodiment of a GULO gene operably linked to an mCherry fluorescent reporter for visualization, and the pAAVS1-NDi-CRISPRi (Gen1) vector that was used.

Pluripotent stem cells were engineered with the mGULO conditional expression construct through conventional approaches, and these mGULO stem cells were differentiated to liver organoids through previously described methods (FIG. 2D). As provided herein, these liver organoids may be cultured with a low dose of bilirubin to promote liver organoid maturation. FIG. 2E depicts brightfield and fluorescence microscopy images of liver organoids expressing the mGULO construct (“mGULO organoids”), where mCherry expression is observed only when doxycycline (Dox) is applied to induce TetOn expression, suggesting the co-expression of mGULO in the liver organoids. When grown in ascorbate-depleted media, the mGULO organoids undergo severe apoptosis, but survivability is rescued when doxycycline is added to induce mGULO expression, which enables the liver organoids to synthesize their own ascorbate (FIG. 2F). The mGULO organoids exhibit a dose dependent correlation between Dox concentration and GULO expression as determined by ELISA and antioxidant levels using the Cellular Antioxidant Assay Kit (abcam), suggesting synthesis of ascorbate (FIG. 2G).

The expression levels of genes involved in oxidative stress response and inflammation were investigated by RT-qPCR in mGULO organoids cultured in ascorbate-depleted media with or without addition of 100 ng/mL Dox (FIG. 2H). Ascorbate-mediated expression of the cytoprotective protein regulator nuclear factor erythroid 2-related factor 2 (NRF2) was elevated in mGULO organoids treated with Dox relative to control and mGULO organoids not treated with Dox. The inflammatory cytokines interleukin 1 beta (ILIB), interleukin 6 (IL6), and tumor necrosis factor alpha (TNFa) were elevated in ascorbate-starved organoids, suggesting significant oxidative stress, whereas this expression of inflammatory factors was rescued to resemble control in mGULO organoids treated with Dox. Similarly, a caspase-3 activity was increased in ascorbate-starved mGULO organoids, suggesting increased apoptosis, but returned to baseline upon exposure to Dox (FIG. 2I).

RNA sequencing (RNA-seq) of mGULO liver organoids treated with Dox revealed increased expression of markers associated liver maturation and/or with the periportal zone of the liver (FIG. 2J). Increased expression in the mGULO organoids was observed for fumarylacetoacetate hydrolase (FAH), albumin (ALB), phenylalanine hydroxylase (PAH), cytochrome P450 3A4 (CYP3A4), carbamoyl-phosphate synthase 1 (CPS1), and homogentisate oxidase (HGD). Generally, expression of genes associated with periportal pathways was observed in mGULO organoids treated with Dox (FIG. 2K).

Microscopy of mGULO organoids treated with Dox and 1 mg/L of bilirubin showed an increasingly complex and irregular lumen shape compared to control organoids without bilirubin and bilirubin-treated organoids (not expressing mGULO) (FIG. 2L). FIG. 2M shows the relative size and circularity of the lumen of mGULO organoids with or without bilirubin compared to control. Furthermore, albumin secretion is significantly increased in mGULO organoids treated with bilirubin (FIG. 2N). mGULO organoids maintain their gross morphology when treated with different concentrations of Dox (10, 100, 1000 ng/mL) (FIG. 2O). The mGULO organoids treated with Dox and low dose bilirubin also exhibit increased CYP3A4 and CYP1A2 activity in response to a rifampicin or omeprazole insult compared to non-mGULO organoids or control (FIG. 2P).

The protein UnaG binds highly specifically to unconjugated bilirubin to form an apoprotein that fluoresces. Other bilirubin-related compounds, including conjugated bilirubin, biliverdin, or urobilin do not have the same ability to make UnaG fluoresce (FIG. 2Q). Therefore, using UnaG, bilirubin conjugation activity was measured in mGULO organoids with or without Dox. A decrease in UnaG fluorescence was observed in Dox-induced mGULO organoids compared to uninduced, indicating that less bilirubin was being bound to UnaG due to conjugation activity (FIG. 2R). Additional information about UnaG can be found in Kumagai et al. A bilirubin-inducible fluorescent protein from eel muscle. Cell (2013) 153 (7): 1602-11, hereby expressly incorporated by reference in its entirety.

Quantifying the total percentage of organoids that conjugate bilirubin using the UnaG assay as well as organoid viability showed that conjugation activity and viability increased with mGULO expression as represented by the concentration of Dox that was applied (FIG. 2S). Overall, this suggests that significant maturation and morphological specification occurs in human liver organoids when enhanced with GULO expression. Accordingly, intracellular ascorbate promotes maturation of fetal-like liver organoids and the introduction of exogenous GULO can be used to improve liver organoid viability, optionally in conjunction with low dose bilirubin.

### Example 4. Hyperbilirubinemia Liver Organoid Models

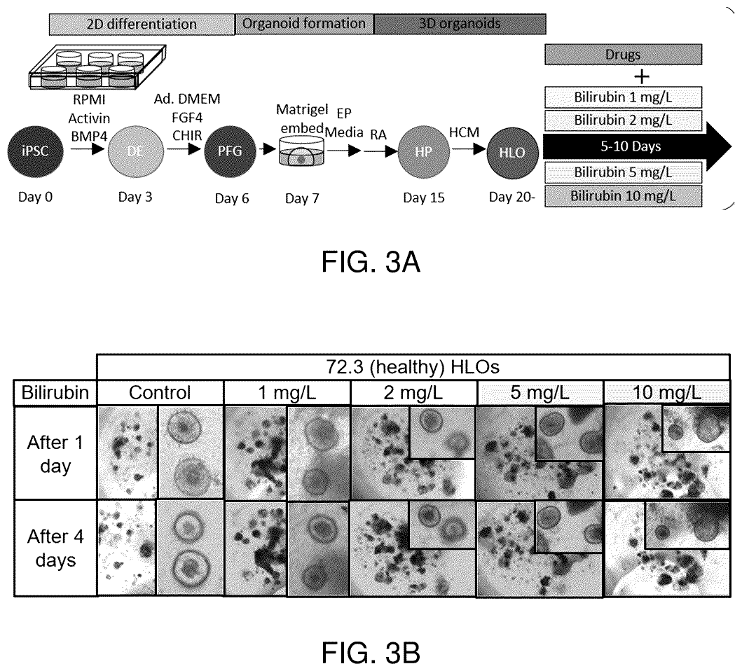

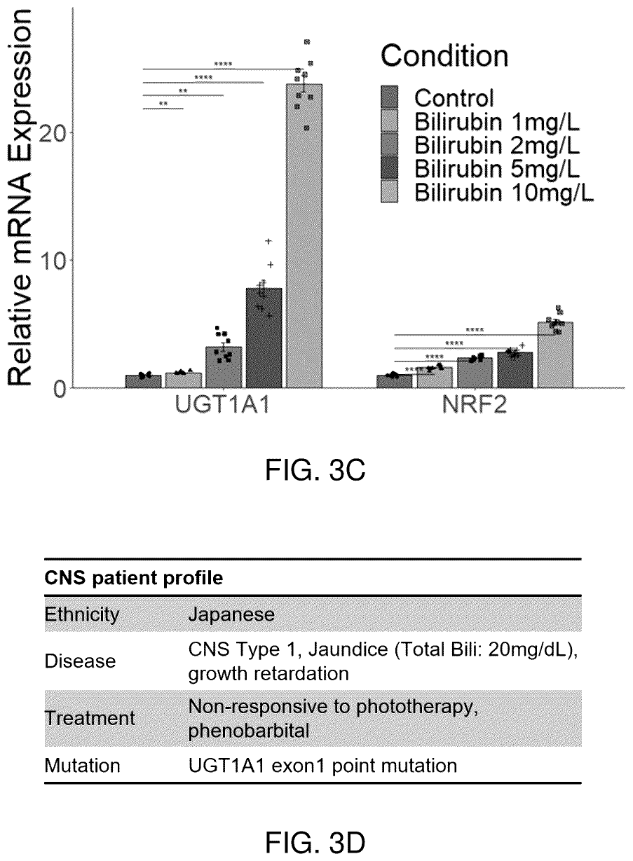

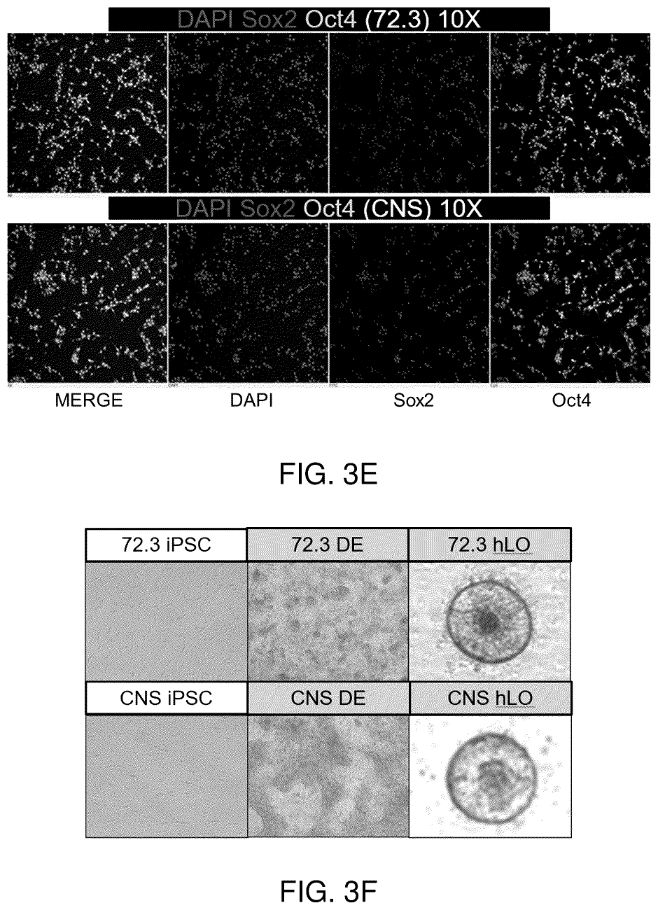

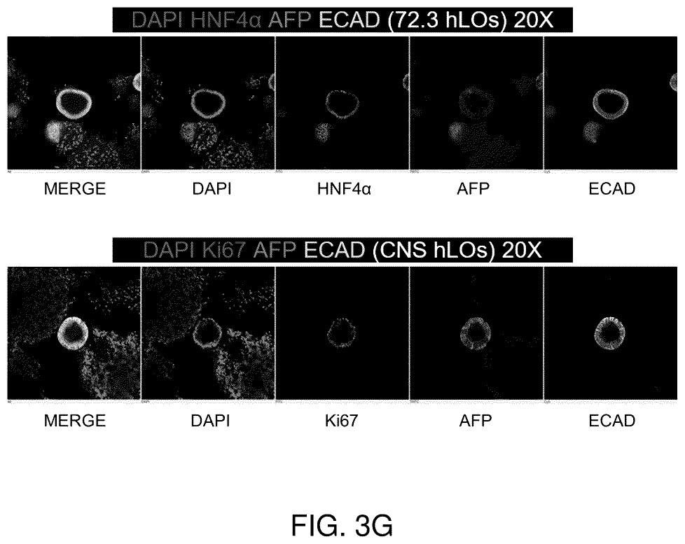

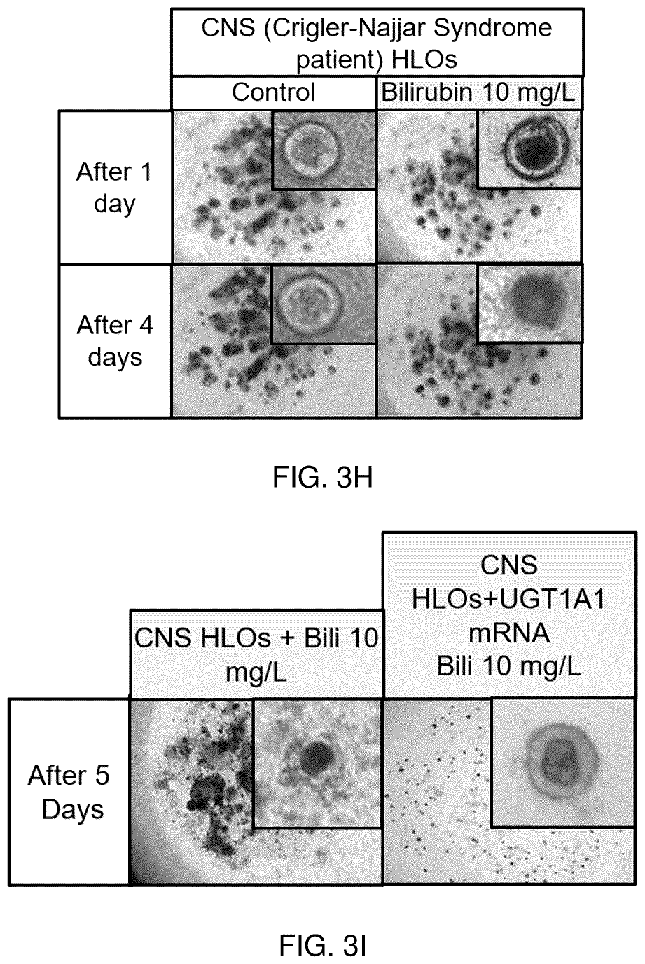

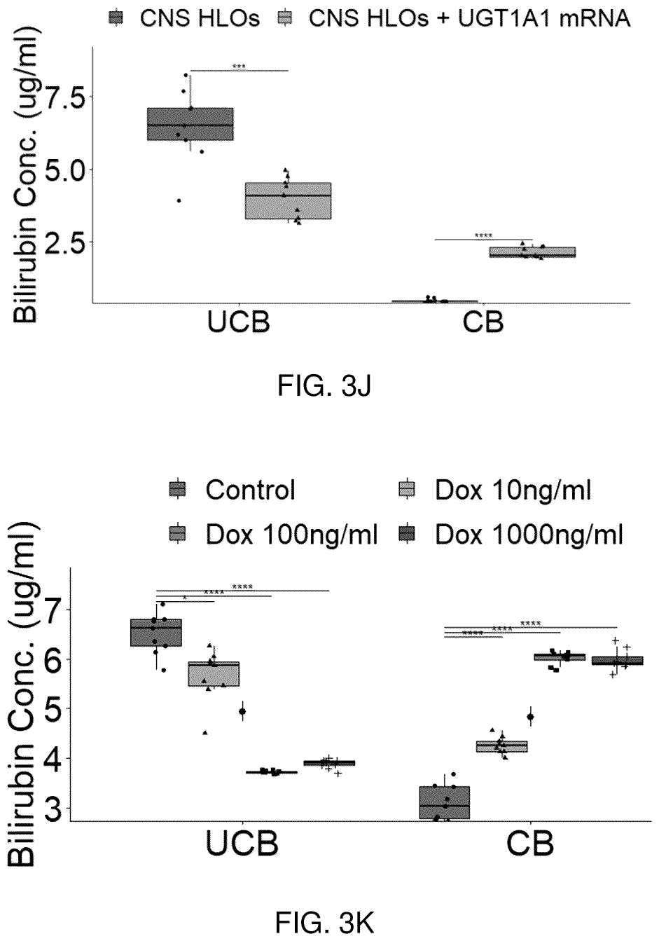

Liver organoids were treated with varying concentrations of bilirubin in culture to induce a hyperbilirubinemic phenotype. FIG. 3A depicts a schematic for this process, where bilirubin was applied at 1, 2, 5, or 10 mg/L to liver organoids that were differentiated from pluripotent stem cells. Liver organoids exhibited significant morphological changes and intracellular accumulation of bilirubin upon exposure to increasing concentrations of bilirubin, indicating that these organoids may be used as a model for hyperbilirubinema (FIG. 3B). RT-qPCR revealed that expression of UDP glucuronosyltransferase family 1 member A1 (UGT1A1) and NRF2 increased corresponding to bilirubin dose (FIG. 3C). UGTIAI is the enzyme that is involved in glucuronic acid conjugation of bilirubin that occurs in the liver, which is needed to render bilirubin water soluble for excretion.

To further investigate the involvement of UGT1A1 in bilirubin clearance and the use of liver organoids for models of diseases associated with bilirubin dysfunction, cells were obtained from a patient diagnosed with Crigler-Najjar syndrome, which is a genetic disorder characterized by the inability to clear bilirubin from the body (FIG. 3D). Genetic sequencing of the patient's cells revealed a c.858C>A (p.Cys280X) nonsense mutation in the UGT1A1 gene.

Induced pluripotent stem cells that were generated from the patient's cells through conventional methods were confirmed to express the canonical pluripotency markers Sox2 and Oct4 (FIG. 3E). These iPSCs were successfully differentiated into definitive endoderm and further into liver organoids according to previously described methods (FIG. 3F), thereby resulting in Crigler-Najjar syndrome liver organoids (“CNS organoids” or “CNS HLOs”). Functional liver phenotype of these CNS HLOs was confirmed by the expression of AFP (FIG. 3G).

Treatment of these CNS HLOs, which are not able to conjugate bilirubin due to the inactive UGT1A1 gene, resulted in liver organoid toxicity when treated with 10 mg/L bilirubin (FIG. 3H). However, this toxicity due to bilirubin was able to be temporarily rescued when exogenous UGT1A1 mRNA was transfected into the CNS HLOs (FIG. 3I). Assay quantification of unconjugated bilirubin (UCB) and conjugated bilirubin (CB) showed that CNS HLOs were unable to conjugate bilirubin, while ectopic expression of UGT1A1 by mRNA delivery enabled some level of bilirubin conjugation (FIG. 3J). Similarly, mGULO expression in normal HLOs also increased bilirubin conjugation (FIG. 3K).

Accordingly, bilirubin is conjugated by UGT1A1 and expression of functional UGT1A1 in a liver organoid model of Crigler-Najjar Syndrome restored bilirubin conjugation function and improved liver organoid survivability. This suggests that these liver organoids may be used to study bilirubin dysfunctions.

### Example 5. UGT1A1 is Negatively Regulated by Glucocorticoid Signaling

Glucocorticoids have been implicated in increased serum bilirubin levels in human. Therefore, the effect of glucocorticoid signaling modulation was investigated using liver organoid models.

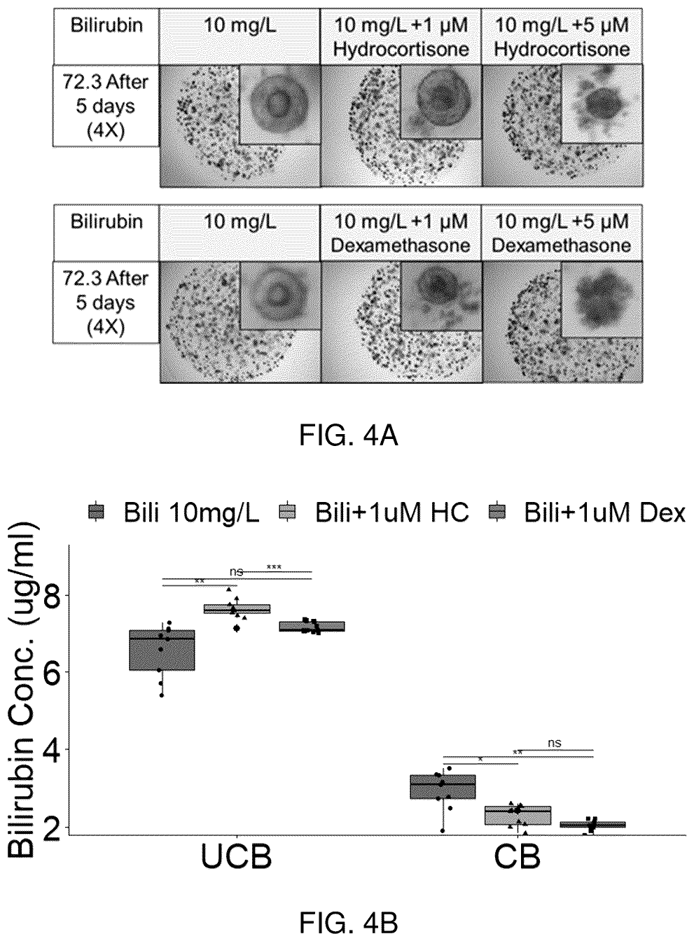

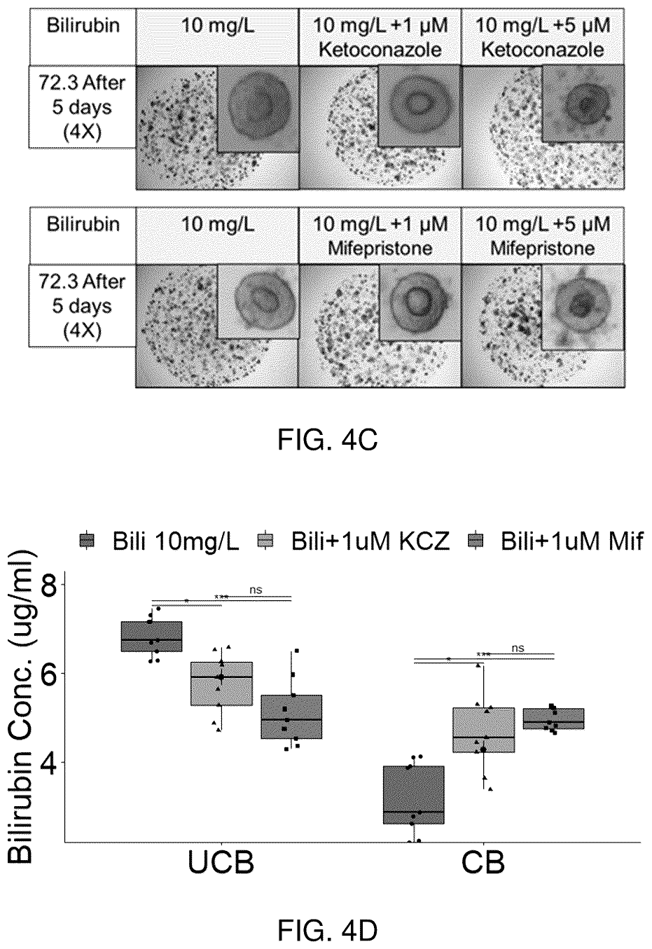

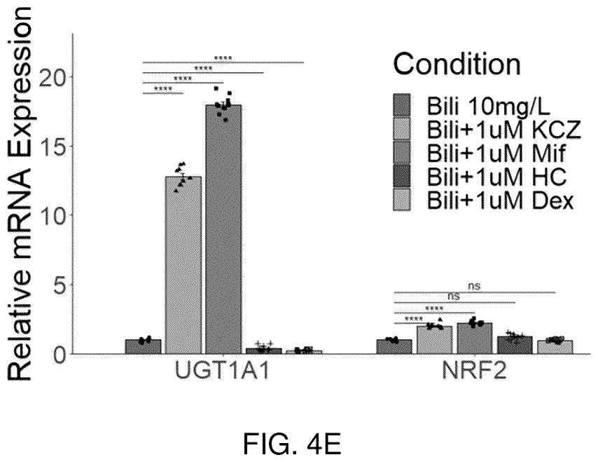

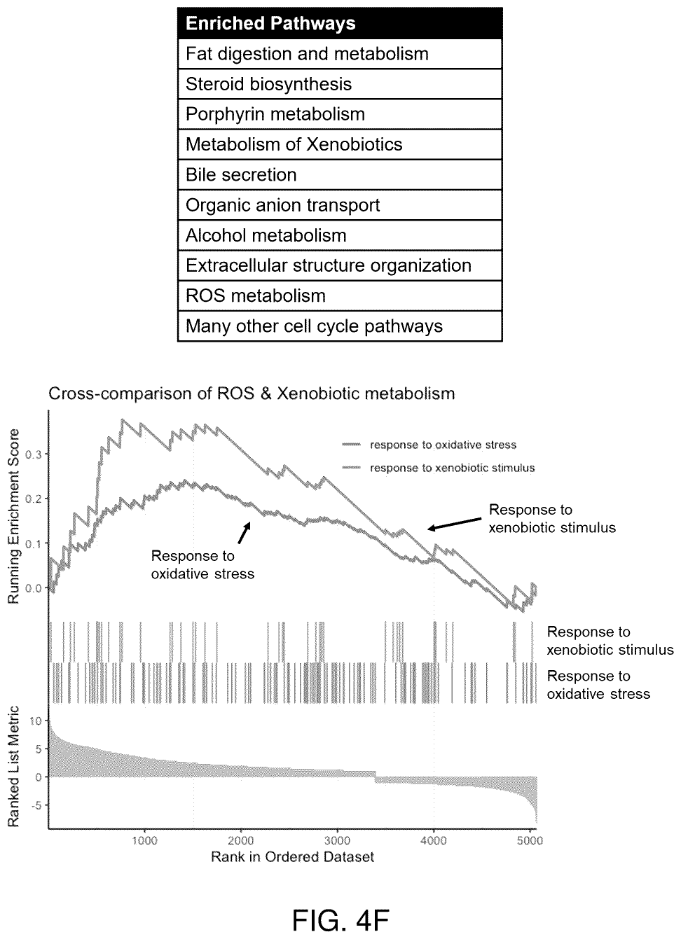

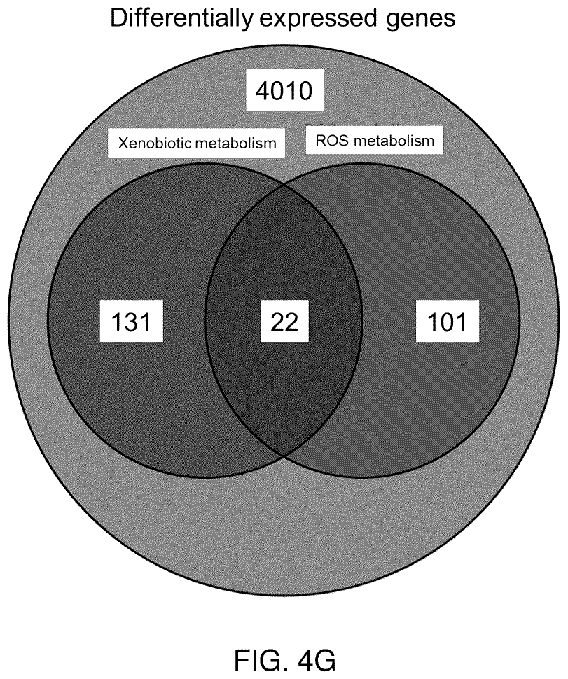

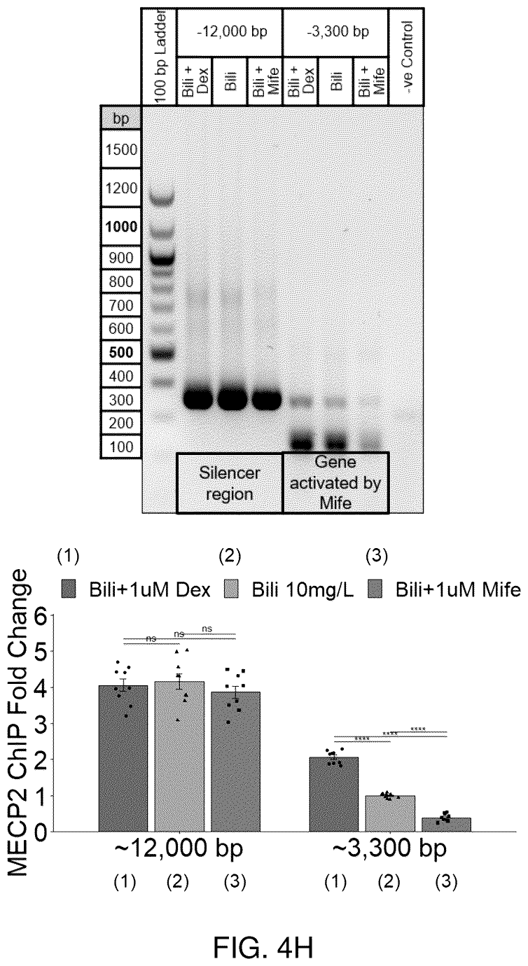

Regular liver organoids (not conditionally expressing mGULO) were cultured in 10 mg/L bilirubin to induce a hyperbilirubinemic phenotype and treated with the glucocorticoid agonists hydrocortisone or dexamethasone, which caused additional organoid toxicity (FIG. 4A) and decreased bilirubin conjugation ability (FIG. 4B). Conversely, hyperbilirubinemic organoids treated with the glucocorticoid antagonists ketoconazole and mifepristone showed normal organoid morphology (FIG. 4C) and increased bilirubin conjugation (FIG. 4D). RT-qPCR revealed that UGT1A1 expression was depressed upon treatment with hydrocortisone or dexamethasone, and elevated upon treatment with ketoconazole or mifepristone (FIG. 4E). NRF2 expression was also improved by ketoconazole or mifepristone treatment. Accordingly, the glucocorticoid pathway was shown to have a role in bilirubin metabolism and clearance in liver organoids.

RNA sequencing and comparison of gene expression between control organoids and those treated with mifepristone showed enrichment of many genes involved in liver function (FIG. 4F). These enriched genes were further categorized as either those that are involved in oxidative stress and/or xenobiotic metabolism (FIG. 4G).

ChIP-PCR and CHIP-qPCR of hyperbilirubinemic organoids treated with mifepristone or dexamethasone showed that methyl CpG binding protein 2 (MECP2) was involved in silencing of the UGT1A1 gene, and treatment with mifepristone reverses this silencing (FIG. 4H).

### Example 6. Orthotopic Transplantation of HLOs Alleviates Hyperbilirubinemia in Rodents

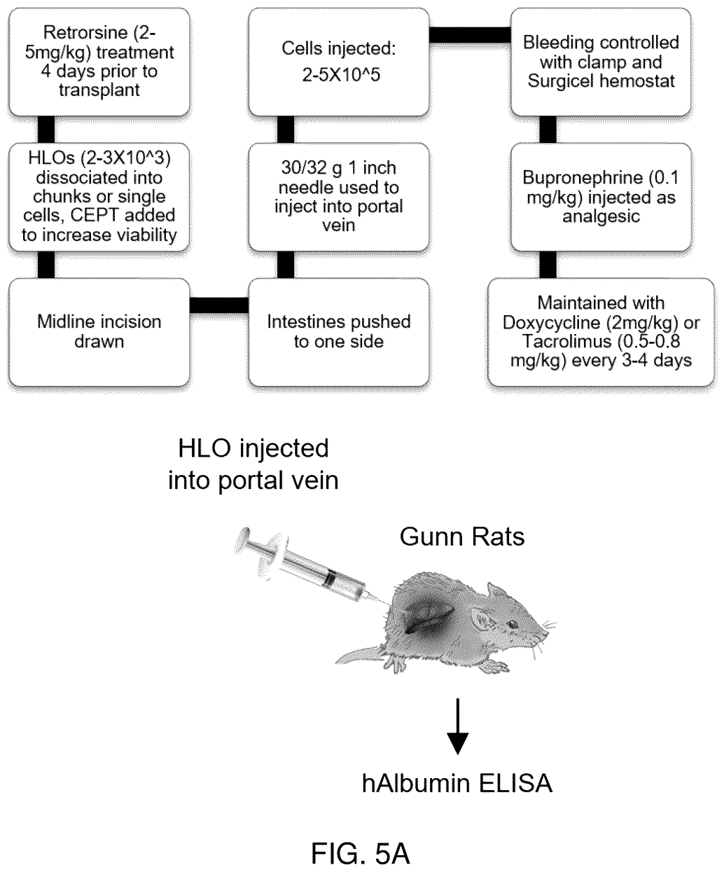

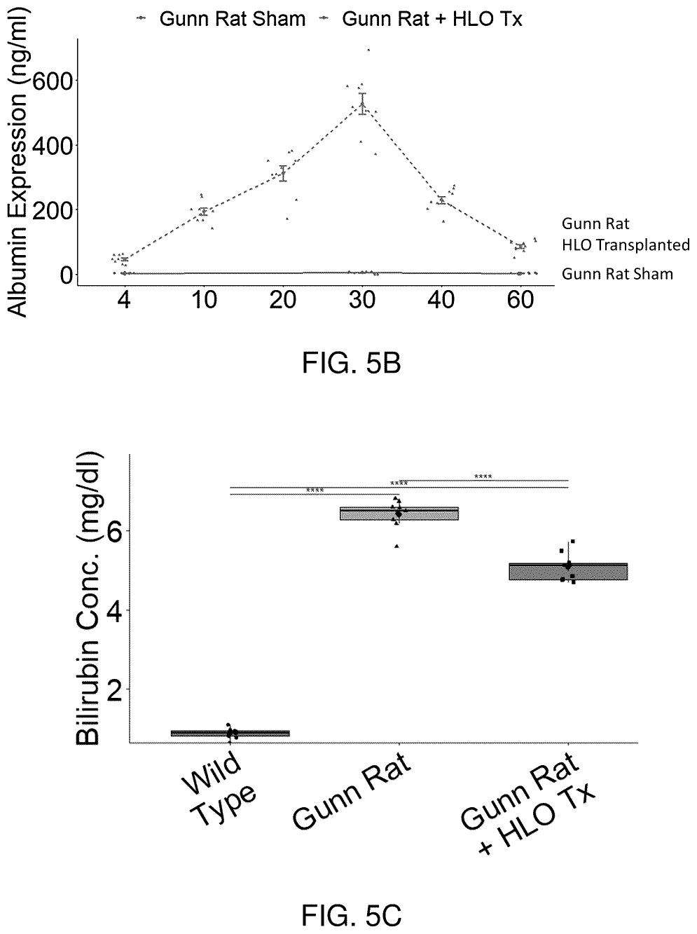

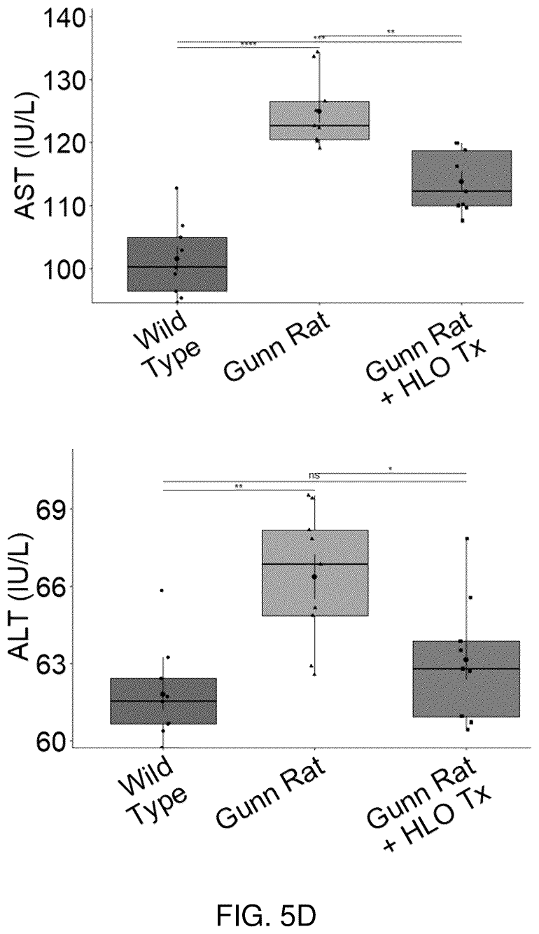

The effect of liver organoid composition administration into an in vivo hyperbilirubinemic rat model was investigated. Gunn rats, which are deficient in all members of the UGTIA family of bilirubin conjugating enzymes, exhibit lifelong unconjugated hyperbilirubinemia. FIG. 5A depicts an exemplary schematic (also described in Example 1) for using human liver organoid compositions to restore bilirubin conjugation ability to Gunn rat models.

FIG. 5B shows elevated albumin production in Gunn rats transplanted with HLOs, suggesting increased liver function. FIG. 5C shows decreased serum bilirubin concentration in HLO-transplanted rats, suggesting an improvement in bilirubin conjugation and clearance. FIG. 5D show decreased serum concentrations of aspartate aminotransferase (AST) and alanine aminotransferase (ALT) in HLO-transplanted rats, where increased levels of AST and ALT is indicative of liver damage, suggesting that the HLO-transplanted rats exhibit healthier liver function.

In at least some of the previously described embodiments, one or more elements used in an embodiment can interchangeably be used in another embodiment unless such a replacement is not technically feasible. It will be appreciated by those skilled in the art that various other omissions, additions and modifications may be made to the methods and structures described herein without departing from the scope of the claimed subject matter. All such modifications and changes are intended to fall within the scope of the subject matter, as defined by the appended claims.

With respect to the use of substantially any plural and/or singular terms herein, those having skill in the art can translate from the plural to the singular and/or from the singular to the plural as is appropriate to the context and/or application. The various singular/plural permutations may be expressly set forth herein for sake of clarity.

It will be understood by those within the art that, in general, terms used herein, and especially in the appended claims (e.g., bodies of the appended claims) are generally intended as “open” terms (e.g., the term “including” should be interpreted as “including but not limited to,” the term “having” should be interpreted as “having at least,” the term “includes” should be interpreted as “includes but is not limited to,” etc.). It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to embodiments containing only one such recitation, even when the same claim includes the introductory phrases “one or more” or “at least one” and indefinite articles such as “a” or “an” (e.g., “a” and/or “an” should be interpreted to mean “at least one” or “one or more”); the same holds true for the use of definite articles used to introduce claim recitations. In addition, even if a specific number of an introduced claim recitation is explicitly recited, those skilled in the art will recognize that such recitation should be interpreted to mean at least the recited number (e.g., the bare recitation of “two recitations,” without other modifiers, means at least two recitations, or two or more recitations). Furthermore, in those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). In those instances where a convention analogous to “at least one of A, B, or C, etc.” is used, in general such a construction is intended in the sense one having skill in the art would understand the convention (e.g., “a system having at least one of A, B, or C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.”

In addition, where features or aspects of the disclosure are described in terms of Markush groups, those skilled in the art will recognize that the disclosure is also thereby described in terms of any individual member or subgroup of members of the Markush group.

As will be understood by one skilled in the art, for any and all purposes, such as in terms of providing a written description, all ranges disclosed herein also encompass any and all possible sub-ranges and combinations of sub-ranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” and the like include the number recited and refer to ranges which can be subsequently broken down into sub-ranges as discussed herein. Finally, as will be understood by one skilled in the art, a range includes each individual member. Thus, for example, a group having 1-3 articles refers to groups having 1, 2, or 3 articles. Similarly, a group having 1-5 articles refers to groups having 1, 2, 3, 4, or 5 articles, and so forth.

While various aspects and embodiments have been disclosed herein, other aspects and embodiments will be apparent to those skilled in the art. The various aspects and embodiments disclosed herein are for purposes of illustration and are not intended to be limiting, with the true scope and spirit being indicated by the following claims.

All references cited herein, including but not limited to published and unpublished applications, patents, and literature references, are incorporated herein by reference in their entirety and are hereby made a part of this specification. To the extent publications and patents or patent applications incorporated by reference contradict the disclosure contained in the specification, the specification is intended to supersede and/or take precedence over any such contradictory material.

### REFERENCES

Ang, Lay Teng, et al. A roadmap for human liver differentiation from pluripotent stem cells. Cell reports (2018) 22 (8): 2190-2205.
Brosch et al. Epigenomic map of human liver reveals principles of zonated morphogenic and metabolic control. Nat. Comm. (2018) 9 (1): 4150.
Buning et al. Higher hydrocortisone dose increases bilirubin in hypopituitary patients-results from an RCT. Eur. J. Clin. Invest. (2016) 46 (5): 475-80.
Creeden et al. Bilirubin as a metabolic hormone: the physiological relevance of low levels. Am. J. Physiol. Endocrinol. Metab. (2021) 320 (2): E191-E207.
Harrison et al. Liver Organoids: Recent Developments, Limitations and Potential. Front. Med. (Lausanne) (2021) 8:574047.
Steeg et al. Complete OATP1B1 and OAP1B3 deficiency causes human Rotor syndrome by interrupting conjugated bilirubin reuptake into the liver. J. Clin. Invest. (2012) 122 (2): 519-28.
Strauss et al. Crigler-Najjar Syndrome Type 1: Pathophysiology, Natural History, and Therapeutic Frontier. Hepatology (2020) 71 (6): 1923-1939.
Teixeira et al. Neonatal Vitamin C and Cysteine Deficiencies Program Adult Hepatic Glutathione and Specific Activities of Glucokinase, Phosphofructokinase, and Acetyl-CoA Carboxylase in Guinea Pigs' Livers. Antioxidants (Basel) (2021) 10 (6): 953.
Traber & Stevens. Vitamins C and E: beneficial effects from a mechanistic perspective. Free Radic. Biol. Med. (2011) 51 (5): 1000-13.
Zhang et al. Human iPSC-Derived Posterior Gut Progenitors Are Exapdable and Capable of Forming Gut and Liver Organoids. Stem Cell Reports (2018) 10 (3): 780-793.

1. A method of maturing a fetal-like liver organoid, comprising contacting a fetal-like liver organoid with a low/first concentration of bilirubin, thereby maturing the fetal-like liver organoid to a mature liver organoid.

2. The method of claim 1, wherein the low/first concentration of bilirubin is a human fetal physiological concentration of bilirubin.

3. The method of claim 1, wherein the low/first concentration of bilirubin is 0.1 to 3 mg/L.

4. The method of claim 1, wherein the mature liver organoid exhibits luminal projections that resemble bile canaliculi.

5. The method of claim 1, wherein the mature liver organoid expresses reduced levels of AFP, CDX2, NANOG, or any combination thereof, relative to the fetal-like liver organoid.

6. The method of claim 1, wherein the mature liver organoid expresses increased levels of ALB, SLC4A2, or HO-1, or any combination thereof, relative to the fetal-like liver organoid.

7. The method of claim 1, wherein the mature liver organoid expresses CYP2E1, CYP7A1, PROX1, MRP3, MRP3, or OATP2, or any combination thereof.

8. The method of claim 1, wherein the mature liver organoid exhibits increased CYP3A4 and CYPIA2 activity relative to the fetal-like liver organoid.

9. The method of claim 1, wherein the mature liver organoid comprises a functional L-gulonolactone oxidase (GULO) protein and/or a gene or mRNA, or both, that encodes for the functional GULO protein, whereby the mature liver organoid is able to synthesize ascorbate.

10. The method of claim 9, wherein the functional GULO protein is murine GULO (mGULO).

11. The method of claim 9, wherein the gene that encodes for the functional GULO protein is conditionally expressed.

12. The method of claim 9, wherein the mature liver organoid is engineered with the gene that encodes for the functional GULO protein using CRISPR.

13. The method of claim 9, wherein the gene or mRNA, or both, that encodes for the functional GULO protein is introduced to the mature liver organoid by transfection.

14. The method of claim 9, wherein the mature liver organoid comprising the functional GULO protein expresses increased levels of NRF2 relative to the fetal-like liver organoid or a mature liver organoid that does not comprise the functional GULO protein.

15. The method of claim 9, wherein the mature liver organoid comprising the functional GULO protein expresses reduced levels of ILIB, IL6, or TNFa, or any combination thereof, relative to the fetal-like liver organoid or a mature liver organoid that does not comprise the functional GULO protein.

16. The method of claim 9, wherein the mature liver organoid comprising the functional GULO protein exhibits reduced caspase-3 activity relative to the fetal-like liver organoid or a mature liver organoid that does not comprise the functional GULO protein.

17. The method of claim 9, wherein the mature liver organoid comprising the functional GULO protein expresses increased levels of ALB relative to the fetal-like liver organoid or a mature liver organoid that does not comprise the functional GULO protein.

18. The method of claim 9, wherein the mature liver organoid comprising the functional GULO protein resembles periportal liver tissue and expresses periportal liver markers.

19. The method of claim 18, wherein the periportal markers comprise FAH, ALB, PAH, CPS1, HGD, or any combination thereof.

20. The method of claim 9, wherein the mature liver organoid comprising the functional GULO protein exhibits increased CYP3A4 and CYPIA2 activity relative to the fetal-like liver organoid or a mature liver organoid that does not comprise the functional GULO protein.

21. The method of claim 9, wherein the mature liver organoid comprising the functional GULO protein exhibits increased bilirubin conjugation activity relative to the fetal-like liver organoid or a mature liver organoid that does not comprise the functional GULO protein.

22. The method of claim 9, wherein the mature liver organoid comprising the functional GULO protein exhibits increased viability in culture relative to the fetal-like liver organoid or a mature liver organoid that does not comprise the functional GULO protein.

23. The method of claim 1, wherein the fetal-like liver organoid is contacted with the low/first concentration of bilirubin in a hepatocyte culture medium.

24. The method of claim 23, wherein the hepatocyte culture medium comprises hepatocyte growth factor, oncostatin M, dexamethasone, or any combination thereof.

25. The method of claim 1, wherein the mature liver organoid is human.

26. The method of claim 1, wherein the mature liver organoid comprises an inactive UGT1A1 gene, wherein the mature liver organoid is a model for Crigler-Najjar Syndrome.

27. The method of claim 1, wherein the fetal-like liver organoid has been differentiated from pluripotent stem cells.

28. The method of claim 27, wherein the pluripotent stem cells comprise a functional GULO protein and/or a gene or mRNA, or both, that encodes for the functional GULO protein, whereby the pluripotent stem cells are able to synthesize ascorbate.

29. The method of claim 1, wherein the fetal-like liver organoid has been made according to a method comprising:
a) contacting definitive endoderm cells (DE) with an FGF signaling pathway activator and a Wnt signaling pathway activator for a first period of time;
b) contacting the cells of step a) with the FGF signaling pathway activator, the Wnt signaling pathway activator, and a retinoic acid (RA) signaling pathway activator for a second period of time, thereby differentiating the DE to posterior foregut cells; and
c) embedding the posterior foregut cells in a basement membrane matrix and culturing the posterior foregut spheroids for a third period of time to differentiate the posterior foregut cells to the fetal-like liver organoid.

30. A method of producing a hyperbilirubinemia liver organoid, comprising contacting a liver organoid with a high/second concentration of bilirubin, thereby forming the hyperbilirubinemia liver organoid.

31. The method of claim 30, wherein the high/second concentration of bilirubin is 2 to 20 mg/L.

32. The method of claim 30, wherein the hyperbilirubinemia liver organoid expresses elevated levels of UGT1A1 or NRF2, or both, relative to a liver organoid not treated with a high/second concentration of bilirubin.

33. The method of claim 30, further comprising contacting the hyperbilirubinemia liver organoid with a glucocorticoid antagonist to reduce the hyperbilirubinemia of the hyperbilirubinemia liver organoid.

34. The method of claim 30, wherein contacting the hyperbilirubinemia liver organoid with the glucocorticoid antagonist increases expression of UGT1A1 and NRF2, and increases bilirubin conjugation activity in the hyperbilirubinemia liver organoid.

35. The method of claim 34, wherein the glucocorticoid antagonist is ketoconazole, mifepristone, or both, or is ketoconazole, mifepristone, metyrapone, aminoglutethimide, or any combination thereof.

36. The method of claim 30, wherein the hyperbilirubinemia liver organoid comprises a functional GULO protein and/or a gene or mRNA, or both, that encodes for the functional GULO protein, wherein the hyperbilirubinemia liver organoid is able to synthesize ascorbate.

37. The method of claim 36, wherein the functional GULO protein is mGULO.

38. The method of claim 36, wherein the gene that encodes for the functional GULO protein is conditionally expressed.

39. The method of claim 30, wherein the hyperbilirubinemia liver organoid comprises an inactive UGT1A1 gene, wherein the hyperbilirubinemia liver organoid is a model for Crigler-Najjar Syndrome.

40.-74. (canceled)
